(19) United States
(12) Patent Application Publication
(10) Pub. No.: US 2010/0004168 A1
(43) Pub. Date: Jan. 7, 2010

(54) Use of Crystallin For The Modulation of Angiogenesis

(75) Inventors: Peter Louis Gehlbach, HUnt Valley, MD (US); Debasish Sinha, Clarksville, MD (US); Stacey Hose, Clear Spring, MD (US); Samuel J. Zigler, JR., Middletown, MD (US); Marisol Cano, Owings Mills, MD (US)

Correspondence Address: EDWARDS ANGELL PALMER & DODGE LLP, P.O. BOX 55874, BOSTON, MA, 02205, US

(73) Assignee: The Johns Hopkins University, Baltimore, MD (US)

(21) Appl. No.: 12/309,026

(22) PCT Filed: Jun. 30, 2006
(86) PCT No.: PCT/US2006/026450

§ 371 Date: Sep. 14, 2009

(51) Int. Cl. A61K 38/16; C07H 21/04; A61K 31/7088; C12N 5/06; C12Q 1/68; C12Q 1/02

(52) U.S. Cl. 514/12; 536/24.5; 514/44 A; 435/325; 435/6; 435/29

(57) ABSTRACT

The present invention features therapeutic and prophylactic compositions and methods for modulating a blood vessel by altering angiogenesis, vasculogenesis, blood vessel stabilization, regression, persistence, or remodeling.

### BACKGROUND OF THE INVENTION

Vasculogenesis, the de novo formation of blood vessels from vascular precursor cells, angiogenesis, the development of new blood vessels from existing vascular beds, and vascular remodeling, morphological changes that occur in blood vessels, are normal physiologic processes that are essential to development during embryogenesis, wound healing, the development of compensatory collateral blood vessels in response to disease, reproductive cycling of uterine lining and other functions. Pathological angiogenesis or pathological neovascularization refers to the development of abnormal blood vessels that supply solid tumors, vascularized benign and malignant malformations, or blood vessel formation in response to disease states such as inflammation, ischemia and other pathologies. Pathological neovascularization is a significant component of disease in nearly all organ systems. For example, in opthalmology pathological ocular neovascularization is a shared and blinding determinant of proliferative diabetic retinopathy, exudative age-related macular degeneration, retinopathy of prematurity and the advanced stages of the occlusive retinal vasculopathies as well as other retinal vascular diseases. Collectively, conditions associated with ocular neovascularization represent the most common causes of blindness in the developed World.

The ability to limit pathological angiogenesis has broad clinical therapeutic application including but not limited to, treatment of cancer, ophthalmic disease, dermatologic disease, rheumatic disease and other diseases where abnormal blood vessel growth is present. The ability to increase blood vessel formation has similarly broad potential applications, including but not limited to, ischemic heart disease other organ ischemia or ischemic limbs as well as ischemic CNS disease.

### SUMMARY OF THE INVENTION

As described below, the present invention features therapeutic and prophylactic compositions and methods for modulating a blood vessel by altering angiogenesis, vasculogenesis, blood vessel stabilization, regression, persistence, or remodeling.

In one aspect, the invention generally features a method for modulating a blood vessel in a subject (e.g., mammal, such as a human patient) in need thereof. The method involves contacting a cell (e.g., a cell of a tissue or organ containing blood vessels) of the subject with a crystallin polypeptide, biologically active fragment, or mimetic thereof, thereby modulating the blood vessel, for example, by increasing or decreasing angiogenesis, vasculogenesis, blood vessel stabilization, regression, persistence, or remodeling. In one embodiment, the method increases or decreases blood vessel formation relative to a reference (e.g., an untreated control tissue or organ). In another embodiment, the method stabilizes or remodels a blood vessel in a tissue or organ relative to an untreated control tissue or organ.

In another aspect, the invention provides a method for decreasing (e.g., by 5%, 10%, 25%, 50%, 75%, 85%, or 95%) angiogenesis in a subject in need thereof. The method involves contacting a cell of the subject with an agent that inhibits the expression or biological activity of a crystallin polypeptide, biologically active fragment, or mimetic thereof.

In yet another aspect, the invention features a method of treating pathological neovascularization (e.g., ocular neovascularization of the iris or retinal choroidal, blood vessels to solid tumors or other neoplasias, vascular malformations, both benign and malignant; vascular abnormalities in development, such as hemangiomas, vascular malformations or the failure to develop normal structures related to abnormal blood vessel development) in a subject. The method involves administering to the subject an agent (e.g., inhibitory nucleic acid molecule, crystallin polypeptide, or biologically active fragment or mimetic thereof, or a nucleic acid molecule encoding same) that decreases angiogenesis in the subject, thereby treating pathological neovascularization in the subject. In one embodiment, the method decreases angiogenesis in a tissue or organ of the subject by at least 5% compared to an untreated control tissue or organ. In other embodiments, the tissue is a neoplastic tissue. In yet other embodiments, the tissue or organ is any one or more of brain, nervous tissue, eye, ocular tissue, heart, cardiac tissue, and skeletal muscle tissue bladder, bone, brain, breast, cartilage, nervous tissue, esophagus, fallopian tube, heart, pancreas, intestines, gallbladder, kidney, liver, lung, ovaries, prostate, skeletal muscle, skin, spinal cord, spleen, stomach, testes, thymus, thyroid, trachea, urogenital tract, ureter, urethra, and uterus.

In another aspect, the invention features a method for treating pathological ocular neovascularization in a subject in need thereof. The method involves administering to the subject an effective amount of an agent that decreases angiogenesis in an ocular tissue thereby treating pathological ocular neovascularization in the subject.

In another aspect, the invention features a method for increasing blood vessel formation in a tissue or organ (e.g., for the treatment of peripheral vascular or coronary vascular disease, birth defects, vascular insufficiency, failure to develop collateral blood vessels in response to stress, such as ischemia). The method involves contacting a cell of the tissue or organ with a crystallin polypeptide, biologically active fragment thereof, or mimetic thereby increasing blood vessel formation in the tissue or organ.

In another aspect, the invention features a method for stabilizing or remodelling a blood vessel in a tissue or organ (e.g., for the treatment of cancer, arthritis, atherosclerosis, restenosis after angioplasty, systemic and pulmonary hypertension, atherosclerosis, embryonic or fetal development, or vascular response to common or atypical disease). The method involves contacting a cell of the tissue or organ with a crystallin polypeptide, biologically active fragment, or mimetic thereof, thereby stabilizing a blood vessel in the subject.

In another aspect, the invention features a method for increasing blood vessel formation or stabilizing or remodeling a blood vessel in a tissue or organ. The method involves contacting a cell of the tissue or organ with a nucleic acid molecule encoding a crystallin polypeptide or biologically active fragment thereof, thereby increasing blood vessel formation or stabilizing a blood vessel in a tissue or organ.

In another aspect, the invention features an inhibitory nucleic acid molecule that specifically binds at least a fragment of a nucleic acid molecule encoding a crystallin polypeptide and decreases the expression of the crystallin polypeptide. In one embodiment, the inhibitory nucleic acid molecule is an siRNA, an antisense oligonucleotide, an shRNA, or a ribozyme.

In another aspect, the invention features an aptamer that specifically binds at least a fragment of a crystallin polypeptide and decreases a biological activity of the crystallin polypeptide.

In a related aspect, the invention features a vector containing a nucleic acid molecule encoding the inhibitory nucleic acid molecule of any previous aspect, wherein the inhibitory nucleic acid molecule is positioned for expression. In one embodiment, the nucleic acid molecule is operably linked to a promoter suitable for expression in a mammalian cell.

In another related aspect, the invention features a host cell containing the nucleic acid molecule of any previous aspect. In one embodiment, the cell is a human cell (e.g., a cell in vitro or in vivo).

In another aspect, the invention features a pharmaceutical composition for modulating a blood vessel in a subject containing an effective amount of a crystallin polypeptide, biologically active fragment, or mimetic thereof in a pharmaceutically acceptable excipient.

In another aspect, the invention features a pharmaceutical composition for modulating a blood vessel in a subject containing an effective amount of an inhibitory nucleic acid molecule of any previous aspect that reduces the expression of a crystallin polypeptide in a pharmaceutically acceptable excipient.

In yet another aspect, the invention features a pharmaceutical composition for modulating a blood vessel in a subject containing an effective amount of an aptamer that specifically binds a crystallin polypeptide or biologically active fragment thereof in a pharmaceutically acceptable excipient.

In yet another aspect, the invention features a pharmaceutical composition containing an effective amount of a vector containing a nucleic acid molecule encoding a crystallin polypeptide or biologically active fragment in a pharmaceutically acceptable excipient, wherein expression of the polypeptide in the cell is capable of modulating a blood vessel.

In another aspect, the invention features a kit for modulating blood vessel formation in a subject in need thereof, the kit containing an effective amount of a crystallin polypeptide or biological fragment thereof and directions for the use of said polypeptide for modulating a blood vessel.

In another aspect, the invention features a kit for modulating blood vessel formation in a subject in need thereof, the kit containing an effective amount of a nucleic acid molecule encoding a crystallin polypeptide or biological fragment thereof and directions for the use of said nucleic acid molecule for modulating a blood vessel formation.

In another aspect, the invention features a kit for decreasing angiogenesis in a subject in need thereof, the kit containing an effective amount of inhibitory nucleic acid molecule or a vector encoding said nucleic acid molecule and directions for the use of said inhibitory nucleic acid molecule or vector to decrease angiogenesis in a subject.

In yet another aspect, the invention features a method of identifying a compound that modulates blood vessel formation. The method involves contacting a cell that expresses a crystallin nucleic acid molecule with a candidate compound, and comparing the level of expression of the nucleic acid molecule in the cell contacted by the candidate compound with the level of expression in a control cell not contacted by the candidate compound, wherein an alteration in expression of the crystallin nucleic acid molecule identifies the candidate compound as a compound that modulates blood vessel formation. In one embodiment, the alteration in expression is a decrease or an increase in transcription. In another embodiment, the alteration in expression is a decrease or an increase in translation.

In another aspect, the invention features a method of identifying a compound that modulates blood vessel formation. The method involves contacting a cell that expresses a crystallin polypeptide with a candidate compound, and comparing the level of expression of the polypeptide in the cell contacted by the candidate compound with the level of polypeptide expression in a control cell not contacted by the candidate compound, wherein an alteration in the expression of the crystallin polypeptide identifies the candidate compound as a compound that modulates blood vessel formation.

In another aspect, the invention features a method of identifying a compound that modulates blood vessel formation. The method involves contacting a cell that expresses a crystallin polypeptide with a candidate compound, and comparing the biological activity of the polypeptide in the cell contacted by the candidate compound with the level of biological activity in a control cell not contacted by the candidate compound, wherein an alteration in the biological activity of the crystallin polypeptide identifies the candidate compound as a candidate compound that modulates blood vessel formation. In one embodiment, wherein the cell (e.g., human cell, such as an endothelial cell is in vitro or in vivo. In other embodiments, the cell is a human umbilical vein endothelial cell (HUVEC) or a Human Retinal Endothelial Cells (HREC). In still other embodiments, the method further involves measuring tube formation in the cell. In yet other embodiments, the alteration in expression is assayed using an immunological assay, an enzymatic assay, or a radioimmunoassay.

In various embodiments of any of the above aspects, the methods decrease or increase angiogenesis, vasculogenesis, blood vessel stabilization, regression, persistence, or remodeling in a tissue or organ of the subject by at least about 5%, 10%, 20%, 30%, 50%, 75%, 85%, 95% or more compared to an untreated control tissue or organ. In other embodiments, the methods of the invention decrease or modulate a blood vessel in a neoplastic tissue. In yet other embodiments, the tissue or organ is any one or more of brain, nervous tissue, eye, ocular tissue, heart, cardiac tissue, and skeletal muscle tissue bladder, bone, brain, breast, cartilage, nervous tissue, esophagus, fallopian tube, heart, pancreas, intestines, gallbladder, kidney, liver, lung, ovaries, prostate, skeletal muscle, skin, spinal cord, spleen, stomach, testes, thymus, thyroid, trachea, urogenital tract, ureter, urethra, uterus, or any other tissue containing a blood vessel. In particular embodiments of any of the above aspects, the method decreases angiogenesis in an ocular tissue by at least 5% compared to an untreated control ocular tissue. In various embodiments of any of the above aspects, the method treats or prevents a vascular disease in a subject, where the disease or disorder is any one or more of proliferative diabetic retinopathy, age-related macular degeneration, retinopathy of prematurity, retinal vascular disease, and occlusive retinal vasculopathy. In one embodiment, the disease or disorder is age-related macular degeneration is the wet or dry form. In various embodiments of the above aspects, the method involves the use of an agent, such as an antibody or an aptamer that binds a crystallin polypeptide, or an inhibitory nucleic acid molecule (e.g., an antisense oligonucleotide, a short interfering RNA (siRNA), ribozyme, or a short hairpin RNA (shRNA)) that decreases the expression of a crystallin polypeptide. In yet other embodiments, the inhibitory nucleic acid molecule comprises a modification that is any one or more of a phosphorothioate backbone, a 2′-OMe sugar modification, and a morpholino backbone structure. In various embodiments, the subject is a mammal, such as a human, that receives a prophylactic or therapeutic agent. In various embodiments, the agent is adminstered during the development of a tissue or organ. In other embodiments, the mammal is contacted prenatally in utero or post-natally. In still other embodiments, the subject has a disease, disorder, or tissue damage and the contacting ameliorates the disease, disorder, or tissue damage. In yet other embodiments, the method decreases angiogenesis in the tissue or organ by at least 5% compared to an untreated control tissue or organ. In other embodiments of any of the above methods, the contacting increases blood vessel formation or stabilizes a blood vessel in a tissue or organ (e.g., bladder, bone, breast, cartilage, esophagus, fallopian tube, pancreas, intestines, gallbladder, kidney, liver, lung, ovaries, prostate, skin, spinal cord, spleen, stomach, testes, thymus, thyroid, trachea, urogenital tract, ureter, urethra, uterus, brain, nervous tissue, eye, ocular tissue, heart, cardiac tissue, and skeletal muscle tissue) of the subject. In still other embodiments, the cell is an endothelial cell, pericyte, muscle cell, neuron or a glial cell. In various embodiments, the cell (e.g., in vivo or in vitro) is contacted during the development of a tissue or organ, for example, prenatally in utero or postnatally. In still other embodiments, the cell is present in a subject that has a disease, disorder, or tissue damage and the contacting ameliorates by increasing or decreasing angiogenesis, vasculogenesis, blood vessel stabilization, regression, persistence, or remodeling the disease, disorder, or tissue damage related to ocular neovascularization of the iris or retinal choroidal, blood vessels to solid tumors or other neoplasias, vascular malformations, both benign and malignant; vascular abnormalities in development, such as hemangiomas, vascular malformations or the failure to develop normal structures related to abnormal blood vessel development, stabilizing or remodelling a blood vessel in a tissue or organ (e.g., for the treatment of cancer, arthritis, atherosclerosis, restenosis after angioplasty, systemic and pulmonary hypertension, atherosclerosis, embryonic or fetal development, or vascular response to common or atypical disease. In still other embodiments of any of the above aspects, the crystallin polypeptide or nucleic acid inhibitor is selected from the group consisting of α, β, γ, αA, αB, β, βγ, γS, ε, δ1, δ2; τ, ζ, μ\acute{η}, ρ, ρB, λπ and their isoforms; and in invertebrates SL11/Lops4, S, Ω/L, J crystallin, and their isoforms, or nucleic acid molecules encoding such polypeptides.

The invention provides methods and compositions for modulating angiogenesis, vasculogenesis, blood vessel stabilization, regression, persistence, or remodeling. Other features and advantages of the invention will be apparent from the detailed description, and from the claims.

### DEFINITIONS

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “crystallin polypeptide” is meant a protein or protein variant, or fragment thereof, that is substantially identical to at least a portion of a crystallin polypeptide and that has a crystallin biological activity (e.g., modulating angiogenesis, vasculogenesis, blood vessel remodeling, regression, or persistence). Virtually any crystallin polypeptide that modulates vascular activity is useful in the methods of the invention, including: αA, αB, β, βγ, γS, ε, δ1, δ2; τ, ζ, μ\acute{η}, ρ, ρB, λπ and their isoforms; and in invertebrates SL11/Lops4, S, Ω/L, J crystallin, and their isoforms. Crystallin polypeptides are reviewed in Piatigorsky J., Gene sharing in lens and cornea: facts and implications. Progress in Retinal and Eye Research. (1998), 17:2, pp. 145-174). In particular embodiments, crystallin polypeptides include, but are not limited to a crystallins (e.g., alpha A, alpha B (GenBank Accession No. NP_{—}000385); beta crystallins (e.g., beta crystallin A1, beta crystallin A2 (GenBank Accession Nos.: AAD45388), beta A3 crystallin GenBank Accession No. NP 005199 crystallin beta B2 (GenBank Accession No. NP_{—}000487), Beta crystallin B3 (GenBank Accession No. P26998), Beta crystallin B2 (GenBank Accession No. P43320); Beta crystallin A4 (GenBank Accession No. P53673), Beta crystallin A2 (GenBank Accession No. P53672), Beta crystallin B1 (GenBank Accession No. P53674), Beta crystallin S (GenBank Accession No. P22914; and gamma crystallins (e.g., Gamma crystallin A (GenBank Accession No. P11844), Gamma crystallin B (GenBank Accession No. P07316), Gamma crystallin C (GenBank Accession No. P07315), Gamma crystallin D (GenBank Accession No. P07320), gamma S crystallin (GenBank Accession No. NP_{—}060011)) gamma E, gammaF, gamma N, and Mu crystallin.

By “crystallin nucleic acid molecule” is meant a polynucleotide encoding a crystallin polypeptide or variant, or fragment thereof.

By “crystallin biological activity” is meant any effect on the vasculature. Specifically, crystallin biological activities include, but are not limited to, increasing or decreasing blood vessel formation, blood vessel stabilization, regression, or persistence, modulation of blood vessel remodelling, or crystallin antibody binding.

By “agent” is meant a compound, polynucleotide, or polypeptide that modulates the expression or biological activity of a target gene or polypeptide.

By “ameliorate” is meant decrease, suppress, attenuate, diminish, arrest, or stabilize the development or progression of a disease.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, for example, hydroxyproline, gamma-carboxyglutamate, and O-phosphoserine, phosphothreonine.

By an “amino acid analog” is meant a compound that has the same basic chemical structure as a naturally occurring amino acid, i.e., a carbon that is bound to a hydrogen, a carboxyl group, an amino group, and an R group (e.g., homoserine, norleucine, methionine sulfoxide, methionine methyl sulfonium), but that contains some alteration not found in a naturally occurring amino acid (e.g., a modified side chain); the term “amino acid mimetic” refers to chemical compounds that have a structure that is different from the general chemical structure of an amino acid, but that function in a manner similar to a naturally occurring amino acid. Amino acid analogs may have modified R groups (for example, norleucine) or modified peptide backbones, but retain the same basic chemical structure as a naturally occurring amino acid. In one embodiment, an amino acid analog is a D-amino acid, a β-amino acid, or an N-methyl amino acid.

Amino acids and analogs are well known in the art. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

By “angiogenesis” is meant the growth of new blood vessels originating from existing blood vessels. Angiogenesis can be assayed by measuring the number of non-branching blood vessel segments (number of segments per unit area), the functional vascular density (total length of perfused blood vessel per unit area), the vessel diameter, or the vessel volume density (total of calculated blood vessel volume based on length and diameter of each segment per unit area).

By “antibody” is meant any immunoglobulin polypeptide, or fragment thereof, having immunogen binding ability.

By “aptamer” is meant an oligonucleotide that binds to a protein.

By “blood vessel formation” is meant the dynamic process that includes one or more steps of blood vessel development and/or maturation. Methods for measuring blood vessel formation and maturation are standard in the art and are described, for example, in Jain et al., (Nat. Rev. Cancer 2: 266-276, 2002).

By “blood vessel remodeling” is meant the structural remodeling and/or differentiation of a blood vessel network. In one embodiment, remodeling alters intimal hyperplasia. In another embodiment, remodelling supports the maturation of an immature blood vessel network. In some embodiments, blood vessel maturation includes the elimination of extraneous vessels.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “an effective amount” is meant the amount of a required to ameliorate the symptoms of a disease relative to an untreated patient. The effective amount of active compound(s) used to practice the present invention for therapeutic treatment of a vascular disease varies depending upon the manner of administration, the age, body weight, and general health of the subject Ultimately, the attending physician or veterinarian will decide the appropriate amount and dosage regimen. Such amount is referred to as an “effective” amount.

An “expression vector” is a nucleic acid construct, generated recombinantly or synthetically, bearing a series of specified nucleic acid elements that enable transcription of a particular gene in a host cell. Typically, gene expression is placed under the control of certain regulatory elements, including constitutive or inducible promoters, tissue-preferred regulatory elements, and enhancers.

By “fragment” is meant a portion (e.g., at least 10, 25, 50, 100, 125, 150, 200, 250, 300, 350, 400, or 500 amino acids or nucleic acids) of a protein or nucleic acid molecule that is substantially identical to a reference protein or nucleic acid and retains at least one biological activity of the reference. In some embodiments the portion retains at least 50%, 75%, or 80%, or more preferably 90%, 95%, or even 99% of the biological activity of the reference protein or nucleic acid described herein.

A “host cell” is any prokaryotic or eukaryotic cell that contains either a cloning vector or an expression vector. This term also includes those prokaryotic or eukaryotic cells that have been genetically engineered to contain the cloned gene(s) in the chromosome or genome of the host cell.

By “inhibitory nucleic acid” is meant a double-stranded RNA, siRNA (short interfering RNA), shRNA (short hairpin RNA), or antisense RNA, or a portion thereof, or a mimetic thereof, that when administered to a mammalian cell results in a decrease (e.g., by 10%, 25%, 50%, 75%, or even 90-100%) in the expression of a target gene. Typically, a nucleic acid inhibitor comprises at least a portion of a target nucleic acid molecule, or an ortholog thereof, or comprises at least a portion of the complementary strand of a target nucleic acid molecule.

The terms “isolated,” “purified,” or “biologically pure” refer to material that is free to varying degrees from components which normally accompany it as found in its native state. Various levels of purity may be applied as needed according to this invention in the different methodologies set forth herein; the customary purity standards known in the art may be used if no standard is otherwise specified.

By “isolated nucleic acid molecule” is meant a nucleic acid (e.g., a DNA, RNA, or analog thereof) that is free of the genes which, in the naturally occurring genome of the organism from which the nucleic acid molecule of the invention is derived, flank the gene. The term therefore includes, for example, a recombinant DNA that is incorporated into a vector, into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule which is transcribed from a DNA molecule, as well as a recombinant DNA which is part of a hybrid gene encoding additional polypeptide sequence.

By “modulation” is meant a change (increase or decrease) in the expression level or biological activity of a gene or polypeptide as detected by standard art known methods such as those described above. As used herein, modulation includes at least about 10% change, 25%, 40%, 50% or a greater change in expression levels (e.g., about 75%, 85%, 95% or more).

By “mimetic” is meant an agent having a structure that is different from the general chemical structure of a reference agent, but that has at least one biological function of the reference.

By “modulating a blood vessel” is meant altering angiogenesis, vasculogenesis, blood vessel stabilization, regression, persistence, or remodeling.

By “nucleic acid” is meant an oligomer or polymer of ribonucleic acid or deoxyribonucleic acid, or analog thereof. This term includes oligomers consisting of naturally occurring bases, sugars, and intersugar (backbone) linkages as well as oligomers having non-naturally occurring portions which function similarly. Such modified or substituted oligonucleotides are often preferred over native forms because of properties such as, for example, enhanced stability in the presence of nucleases.

Specific examples of some nucleic acids envisioned for this invention may contain phosphorothioates, phosphotriesters, methyl phosphonates, short chain alkyl or cycloalkyl intersugar linkages or short chain heteroatomic or heterocyclic intersugar linkages. Also preferred are oligonucleotides having morpholino backbone structures (Summerton, J. E. and Weller, D. D., U.S. Pat. No. 5,034,506). In other preferred embodiments, such as the protein-nucleic acid (PNA) backbone, the phosphodiester backbone of the oligonucleotide may be replaced with a polyamide backbone, the bases being bound directly or indirectly to the aza nitrogen atoms of the polyamide backbone (P. E. Nielsen et al. Science 199: 254, 1997). Other preferred oligonucleotides may contain alkyl and halogen-substituted sugar moieties comprising one of the following at the 2′ position: OH, SH, SCH₃, F, OCN, O(CH₂)ₙNH₂ or O(CH₂)ₙCH₃, where n is from 1 to about 10; C₁ to C₁₀ lower alkyl, substituted lower alkyl, alkaryl or aralkyl; Cl; Br; CN; CF₃; OCF₃; O-, S-, or N-alkyl; O-, S-, or N-alkenyl; SOCH₃; SO₂CH₃; ONO₂; NO₂; N₃; NH₂; heterocycloalkyl; heterocycloalkaryl; aminoalkylamino; polyalkylamino; substituted silyl; an RNA cleaving group; a conjugate; a reporter group; an intercalator; a group for improving the pharmacokinetic properties of an oligonucleotide; or a group for improving the pharmacodynamic properties of an oligonucleotide and other substituents having similar properties. Oligonucleotides may also have sugar mimetics such as cyclobutyls in place of the pentofuranosyl group. Other preferred embodiments may include at least one modified base form. Some specific examples of such modified bases include 2-(amino)adenine, 2-(methylamino)adenine, 2-(imidazolylalkyl)adenine, 2-(aminoalklyamino)adenine, or other heterosubstituted alkyladenines.

By “operably linked” is meant that a first polynucleotide is positioned adjacent to a second polynucleotide that directs transcription of the first polynucleotide when appropriate molecules (e.g., transcriptional activator proteins) are bound to the second polynucleotide.

By “pathological neovascularization” is meant an excess or abnormal formation of blood vessels in a tissue or organ.

By “pathological ocular neovascularization” is meant an excess or abnormal formation of blood vessels in the eye.

By “recombinant” is meant the product of genetic engineering or chemical synthesis. By “positioned for expression” is meant that the polynucleotide of the invention (e.g., a DNA molecule) is positioned adjacent to a DNA sequence that directs transcription and translation of the sequence (i.e., facilitates the production of, for example, a recombinant protein of the invention, or an RNA molecule).

By “reference” is meant a standard or control condition.

By “ribozyme” is meant an RNA that has enzymatic activity, possessing site specificity and cleavage capability for a target RNA molecule. Ribozymes can be used to decrease expression of a polypeptide. Methods for using ribozymes to decrease polypeptide expression are described, for example, by Turner et al., (Adv. Exp. Med. Biol. 465:303-318, 2000) and Norris et al., (Adv. Exp. Med. Biol. 465:293-301, 2000).

By “siRNA” is meant a double stranded RNA. Optimally, an siRNA is 18, 19, 20, 21, 22, 23 or 24 nucleotides in length and has a 2 base overhang at its 3′ end. These dsRNAs can be introduced to an individual cell or to a whole animal; for example, they may be introduced systemically via the bloodstream. Such siRNAs are used to downregulate mRNA levels or promoter activity.

By “specifically binds” is meant a molecule (e.g., peptide, polynucleotide) that recognizes and binds a protein or nucleic acid molecule of the invention, but which does not substantially recognize and bind other molecules in a sample, for example, a biological sample, which naturally includes a protein of the invention.

By “stabilizes” a blood vessel is meant increases the survival or maintenance of the blood vessel in a tissue relative to a control tissue.

By “subject” is meant a mammal, including, but not limited to, a human or non-human mammal, such as a bovine, equine, canine, ovine, or feline.

By “substantially identical” is meant a protein or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and most preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “transformed cell” is meant a cell into which (or into an ancestor of which) has been introduced, by means of recombinant DNA techniques, a polynucleotide molecule encoding (as used herein) a protein of the invention.

By “vascular disease or disorder” is meant any pathology that disrupts the normal function of a blood vessel or that results in excess or abnormal blood vessel formation. Exemplary vascular diseases or disorders include, but are not limited to, atherosclerosis, restenosis, systemic and pulmonary hypertension, intimal hyperplasia, peripheral artery disease, limb ischemia, cancer, arthritis, cardiac ischemia, stroke, proliferative diabetic retinopathy, age-related macular degeneration, retinopathy of prematurity, retinal vascular disease, and occlusive retinal vasculopathy.

By “vasculogenesis” is meant the development of new blood vessels originating from stem cells, angioblasts, or other precursor cells.

### BRIEF DESCRIPTION OF THE DRAWINGS

FIGS. 1A-1C provides a description of the eye and crystallin development FIG. 1A is a schematic diagram of the intraocular vessels. During development of the mammalian eye, the nourishment of the immature lens, retina, and vitreous is ensured by the hyaloid vascular system, including the pupillary membrane, tunica vasculosa lentis, vasa hyaloidea propria, and the hyaloid artery as shown in this schematic diagram. The hyaloid vessels regress by a programmed cell death process during ocular development, pre-natally in humans and within the first few weeks of post-natal development in rodents. In rats, the hyaloid vessels undergo involution, starting at post-natal day 7, when retinal vessels start developing. The transient hyaloid vessels in the rat normally regress within 3 weeks after birth and as the retina matures, the inner part of the retina becomes vascularized by retinal vessels. The mature lens and vitreous remain avascular to provide an optically clear path to the retina. FIG. 1B shows developmental expression of β-crystallins. Immunofluorescent labeling of β-crystallins in the developing eye. DAPI staining for the nucleus. Expression of β-crystallins in the mouse eyes at 12, 16 and 18 embryonic days. β-crystallin expression by 18 embryonic days (arrow) in the retina, as well as in the hyaloid vasculature (*) is evident. While the lens expresses β-crystallins much earlier in development, retinal expression is evident at embryonic day 18. FIG. 1C shows crystallin expression in the developing eye of the mouse: immunoflourescent labeling (green) of Isolectin B4 staining of the hyaloid vasculature (hyaloid artery-arrows, tunica vasculosa lentis-asterisk) in two, nine and twelve day old mice (A, D, and G).

FIGS. 2A-2G are a series of photomicrographs showing the defective regression of embryonic vasculature in Nuc1 mutant rat. FIG. 2A shows that in a wild type animal, the hyaloid artery had completely regressed by 5 weeks of age, showing a normal optic nerve head (ONH). In 5-week-old Nuc1/Nuc1 rats (FIGS. 2B and 2C), the hyaloid artery and adjacent tissue were still present on the surface of the optic nerve head projecting into the vitreous (arrow). Note in FIG. 2C, the thick vessel wall of the artery at higher magnification showing cellular morphology of the retained vessel. FIG. 2D shows representative H & E stained sections from 25-day-old animals show normal lens (L), iris (I), ciliary body (CB), and cornea (C). FIG. 2E shows that the pupillary membrane is still evident in the Nuc1 homozygous animals (arrows), whereas it has fully regressed in the wild type eye (FIG. 2D). Iris hyperplasia was also noted in the Nuc1/Nuc1 eyes (arrowheads). The lens shows abnormal shape and disorganization of structure. FIG. 2F shows the normal eye structure at 120 days in wild-type eyes. In Nuc1 homozygote (FIG. 2G), the ciliary process (arrow) is dragged centrally towards the disrupted lens, resulting in traction, which causes peripheral retinal dragging and folding (arrowhead). Scale bar=0 μm.

FIGS. 3A-3D show crystallin expression in the retina. FIGS. 3A and 3B are histograms showing real-time RT-PCR analysis of total RNA (1 μg) from 25-day-old retina clearly show upregulation of β-crystallins (βA1/A3, γA4, βB1, γB3) and γ-crystallins (γA-γF) in Nuc1 homozygous rats (dark gray bar) compared to wild-type controls (gray bar). Data shown as mean+/−SEM. FIG. 3C is an autoradiograph of 4-12% Bis-Tris Nu-PAGE gel showing that new synthesis of proteins in the 20-30-kD range is markedly increased in Nuc1 retina compared to age-matched wild-type retina. FIG. 3D is a Western blot probed using anti β and γ-crystallin antibodies, clearly identifies β, γ-crystallins as major components in the upregulated protein fraction. This is consistent with the upregulation in mRNA levels in Nuc1 homozygous retina.

FIGS. 4A-4E are photomicrographs showing immunofluorescent staining of persistent transient vessels in Nuc1 and expression of crystallins. FIG. 4A shows a retained hyaloid artery in Nuc1 at post-natal day 25, which shows multiple branches, known as the vasa hyloidea propria, that are configured like the struts of an umbrella or guide ropes of a parachute (arrow). Such a structure has also been reported in human Persistent Fetal Vasculature (PFV) disease. Note, fluorescence indicating presence of γ-crystallin in the retained hyaloid tissue. In rats, the intraocular vessels normally regress within 3 weeks after birth. FIG. 4B shows the persistent pupillary membrane (PM) in Nuc1 shows expression of crystallins (short arrows) in the retained vessels (v). The section was stained with isolectin B4 (large arrows) and Hoechst.] Isolectin 14 has previously been shown to localize rat blood vessels. Hoechst was used for staining nuclei. Note that there was crystallin immunoreactivity also in the lens epithelium (LE) and lens capsule (LC, short arrow) with some scattered staining in the hyperplastic iris (I). Lectin staining was also evident in the anterior part of the iris (large arrows). In the degenerate lens shown in this section, the area beneath the epithelium was devoid of cells (see FIG. 1e). FIG. 4C shows that crystallin staining is discretely localized peripheral to the vessels of the retained hyaloid tissue (arrows) relative to isolectin B4 that stains the vessel wall (FIG. 4D). The merged image (FIG. 4E) demonstrates that the crystallin staining and the lectin staining are discrete with virtually no overlap. In FIGS. 4D and 4E, a portion of image 4C is shown at a slightly higher magnification. For orientation, the lumen of each major vessel visible in these images is marked with an asterisk. Scale bar=50 μm.

FIG. 5A-5D are photomicrographs showing an immunofluorescent analysis of crystallin expression in the normal and Nuc1 homozygous rats during development. Confocal microscopy of 9-day-old normal (FIG. 5A) and Nuc1 homozygous (FIG. 5B) hyaloid artery shows distinct crystallin expression surrounding the vessels (arrows). The normal rat (FIG. 5A) was FITC/Dextran perfused (asterisk) and was counterstained with DAPI (arrowhead). The Nuc1 homozygous hyaloid artery was stained with Isolectin-B4 (FIG. 5B). Note, in both wild-type and Nuc1 homozygous hyaloid artery, crystallin is expressed surrounding the vessel wall. A similar pattern of crystallin expression was also observed in the retinal vasculature during development of both wild-type and Nuc1 homozygous rats. By 20 days, in normal rats (FIG. 5C) and Nuc1 homozygous (FIG. 5D), crystallin expression was mostly localized surrounding the vessels of the inner limiting membrane of the retina (arrows). The rats were perfused with FITC-Dextran (asterisk) and counterstained with DAPI (arrowhead).

FIGS. 6A-6F are photomicrographs showing an immunofluorescent analysis of VEGF and crystallin expression in astrocytes. FIG. 6A shows immunofluorescent labeling of sections from 5-week-old Nuc1 homozygotes with VEGF antibodies shows positive staining surrounding the vessels of the retained hyaloid artery. FIG. 6B shows positive staining using with antibody against GFAP (green). FIG. 6C is a merged image showing co-localization of VEGF and GFAP (arrows). FIG. 6D shows staining with γ-crystallin antibodies. These antibodies show positive staining surrounding the vessels of the retained hyaloid artery of S-week-old Nuc1 homozygotes (red). FIG. 6E shows that the hyaloid vasculature was immunopositive for GFAP (green). FIG. 6F is a merged image of FIGS. 6D and 6E, which reveals co-localization of the γ-crystallin and GFAP immunoreactivity in the cells surrounding the retained vasculature. The nuclei are stained with Hoechst. Scale bar=50 μm .

FIGS. 7A-7F are photomicrographs showing an immunofluorescent analysis of VEGF and Crystallin expression in the retinal astrocytes. FIG. 7A shows a Confocal microscopic analysis of GFAP positive staining at the internal limiting membrane of 5 week old Nuc1 homozygote retina. FIG. 7B shows γ-crystallin also stained positive at the internal limiting membrane and ganglion cells (arrowheads). FIG. 7C is the merged image, which shows co-localization in the internal limiting membrane (arrows). In 5-week-old Nuc1 homozygote, the internal limiting membrane of the retina is positive for both GFAP (FIG. 7D) and VEGF (FIG. 7e) staining. FIG. 7F is a merged image, which shows co-localization (arrows). The nuclei are stained with Hoechst. Scale bar=50 μm.

FIGS. 8A-8F show VEGF and crystallin expression in human PFV disease. PAS staining of human PFV tissue showed centrally dragged retina with rosette formation (FIG. 8A, short arrows) and a retained hyaloid artery projecting into the vitreous chamber (a and b, long arrow). FIG. 8B shows the higher magnification view of the hyaloid artery shown in FIG. 8A. Histologically, the structure is similar to Nuc1 (FIG. 2) although no rosette formation has been observed in Nuc1. FIG. 8C shows immunofluorescent labeling with VEGF shows staining surrounding the vessel wall of the retained hyaloid artery. FIG. 8D shows the same section counterstained with Hoechst to label nuclei. Staining patterns similar to that of VEGF were obtained using antibodies to GFAP (FIG. 8e) and crystallin (FIG. 8F). Note that the staining pattern of GFAP-positive astrocytes, VEGF, and γ-crystallin in PFV are similar to that observed in Nuc1 (FIGS. 4, 6, and 7). Scale bar=50 μm.

FIG. 9 shows VEGF and crystallin expression in cultured astrocytes exposed to 3-nitropropionic acid (3-NP). Human astrocyte cell line (SVG) was treated with 10 μM 3-NP, and cells were harvested for immunostaining with antibodies to VEGF and crystallins after 6, 12, and 24 hours. Photomicrographs (counterstained with DAPI) show that normal astrocytes do not express γ-crystallins or VEGF (top). When cells are exposed to 3-NP for 6 hours, distinct cytoplasmic staining for both γ-crystallin and VEGF has been observed. Fluorescence with both of the antibodies is increased after 12 hours of exposure to 3-NP in culture. By 24 hours, most of the cells have died. Nuclei were counterstained with DAPI.

FIGS. 10A-10H provide histological and morphometric analyses of retinas during the developmental phase, when most of the remodeling occurs. This analysis revealed abnormalities in the Nuc1 homozygous rats. At E16/17, when the inner and the outer neuroblastic layers are formed in the rat, no difference in the organization of the wild-type (FIG. 10A) and Nuc1 homozygote retina (FIG. 10B) was observed. Morphometric analysis indicates that there is no difference in the retinal thickness at this stage (FIG. 10C). By P3, the ganglion cell layer starts to form, as shown in the wild-type (FIG. 10D). While the ganglion cell layer is also formed in Nuc1 homozygotes, the retina appears to be thicker, probably due to hyperplasia as evidenced by increased number of nuclei (FIG. 10E, arrows and FIG. 10F). By P12, following the first apoptotic wave and when the second wave is in progress, the Nuc1 homozygote retina is significantly thicker in all cellular layers (g-i). Scale bar=100 μm.

FIGS. 11A-11C are photomicrographs (FIGS. 11A and B) and a Western blot (FIG. 11C). Double-labeled wild-type (FIG. 11A) and Nuc1 homozygote (FIG. 11b) retina with proliferating cell nuclear antigen (PCNA, light gray) and DAPI (medium gray) at birth. Numerous cells with positively stained nuclei (PCNA and DAPI) are present in both the wild-type and Nuc1 homozygotes and the staining patterns are similar. FIG. 11C is a Western analysis of p27kip1 from SDS-PAGE-separated retina proteins. Similar intensity is seen for wild-type and homozygous Nuc1 samples at both ages tested. P27kip1 is much more abundant at 12 days than at 2 months.

FIGS. 12A-12L are photomicrographs showing immunofluorescent labeling performed on frozen sections of wild-type and Nuc1 homozygous rats at P9, 25, and 87. Monoclonal antibodies to neurofilament 70 (FIGS. 12A-F) and to syntaxin (G-L), specific markers for ganglion cells and amacrine cells, respectively show delayed development in Nuc1 homozygous rats. While ganglion cells are clearly labeled by anti-neurofilament 70 antibody by P9 in wild-type retina (FIG. 12A), antibody staining only became visible around P25 days in Nuc1 homozygotes (FIG. 12E). Both wild-type and Nuc1 homozygous retinas are stained for the antibody at P87 days (FIGS. 12C and 12F). Staining of amacrine cell processes is weak in the Nuc1 retina at 9 days (FIG. 12J) relative to wild-type (FIG. 12G). By 25 days, staining has increased dramatically in the mutant retina and this intensity increases further by 87 days. Note, the increased thickness of the IPL in Nuc1 homozygotes at P25 and P87 days.

FIGS. 13A-13L are photomicrographs showing immunofluorescence labeling performed on frozen sections of wild-type and Nuc1 homozygous rats at P9, 25, and 87. Horizontal cells (arrows) labeled by polyclonal antibodies to calbindin (FIGS. 13A-F) and bipolar cells labeled by antibody to PKC-α (FIGS. 13G-13L) also show delayed development in Nuc1 homozygotes. While horizontal and bipolar cells are clearly labeled by their respective antibodies at P9 in wild-type retina (FIGS. 13A and G), in Nuc1 homozygotes, the antibody staining was not visible until P25 (FIGS. 13E and 13K). Note that in the Nuc1 homozygotes, there is decreased staining of bipolar cells compared with wild-type FIGS. 14A-14E are panels showing that wave-form morphology has diminished a-wave and b-wave amplitudes in homozygous but not heterozygous animals, as compared with wild-type at 10 weeks. Light-adapted wave forms are preserved in all animals FIG. 14A shows ERG amplitude vs. flash intensity (V-log I) curves for the dark-adapted a-wave (FIG. 14B), dark-adapted b-wave (FIG. 14C) and light adapted b-wave (FIG. 14D) in each group of rats: wild-type (open circles, n=6), Nuc1 heterozygotes (filled squares, n=6) and Nuc1 homozygotes (filled triangles, n=5) are presented. Error bars indicate standard deviation. The dark-adapted b-wave amplitudes in the Nuc1 homozygous rats were significantly smaller than the wild-type and the Nuc1 heterozygous rats (P_{—}0.01 at all intensities). The dark adapted a-wave amplitudes of the Nuc1 homozygous rats were smaller at the highest flash intensities relative to the wild-type (P>0.05) and the Nuc1 heterozygous (P_{—}0.05) rats. The light-adapted b-wave amplitudes of the Nuc1 homozygous rats were comparable to the wild-type and the Nuc1 heterozygous rats (P<0.05). FIG. 14E is a graph showing that the dark-adapted ERG b-wave/a-wave ratio of the homozygous Nuc1 rats was smaller than that of the control (P<0.05) and the heterozygous (P<0.05) rats.

FIGS. 15A-15F shows immunolabeling performed on frozen sections using antibodies to rhodopsin kinase 1α (for rod staining), JH455 for s-cones and JH492 for m-cones in 10 week old wild-type and Nuc1 homozygous rats. A marked abnormality in the rod staining pattern of the Nuc1 homozygous rats was observed compared with wild-type (FIGS. 15A and 15B). In the wild-type rat (FIG. 15A), the outer segments (OS) showed intense staining with the rhodopsin antibody with moderate intensity of labeling in the peri-nuclear region of photoreceptor nuclei in the ONL. In the Nuc1 homozygous rats, the rhodopsin labeling was patchy and not evenly distributed in the OS and ONL. The positively labeled M-cones (arrows) appeared increased in number in the Nuc1 homozygous rats as compared with the wild-type rats (FIGS. 15C and 15D). Due to the small number of positively labeled S-cone cells, scattered S-cone cells showed no difference between wild-type and Nuc1 homozygous rats (FIGS. 15E and 15F).

FIGS. 16A-16E show representative H & E stained sections of 10 week old retinas showing normal morphology in the wild-type (FIG. 16A) but in the homozygous Nuc1, the retina is thicker (FIG. 16B) when compared with wild-type ((FIG. 16A) and shows focal retinal detachment ((FIG. 16C). As the Nuc1 homozygous retina ages ((FIGS. 16D and 16E), retinal thinning and traction retinal detachment (rd) with sub-retinal fluid (SRF) and several other abnormalities are observed. These are in 18 months Nuc1 retina ((FIG. 16D) and at higher magnification ((FIG. 16E) ERF, epiretinal fibrosis; PRV, preretinal vasculature.

FIGS. 17A-17L are photomicrographs showing immunofluorescent staining in Muller glial cells labeled with polyclonal antibodies to CRALBP (17A-17F) and GFAP (17G-17L) at P9, 25 and 87 days for both wild-type and Nuc1 homozygous rats. Unlike retinal neurons, Nuc1 homozygotes are positive even at P9 for both CRALBP and GFAP, but the staining is much more intense at both P25 and P87 days (FIGS. 17E and 17F and 17K and 17L) in the mutant retina compared with the wild-type.

FIGS. 18A and 18D show protein expression in wild-type and Nuc1 retinas. FIGS. 18A and 18B show two-dimensional electrophoresis patterns for 2 month old wild-type (FIG. 18A) and Nuc1 (FIG. 18B) retinas. The patterns are grossly similar, but note the dramatic increase in spot 1 in the Nuc1 pattern. This spot was identified by mass spectrometry as GFAP. A number of crystallins were also shown to be increased in the Nuc1 retina (spots 5-8). To confirm and extend the GFAP result samples from 4, 6, and 10 week retinas were ran on ID SDS-PAGE (FIG. 18C). Western analysis of these samples with a GFAP-specific antibody confirmed the marked increase in GFAP in the Nuc1 retina; this increase appears to occur only after the age of 4 weeks. The spots identified are: 1—GFAP, 2—α-enolase, 3—aldolase, 4—ribonucleoprotein A2/B1, 5—β2-crystallin, 6—αA-crystallin, 7—βA4-crystallin, 8—αB-crystallin.

FIGS. 19A-19H are photomicrographs showing a fluorescence microscopic analysis of flatmounts from FITC-dextran perfused wild-type and Nuc1 homozygous retinas. These studies show that the vascularization of the Nuc1 homozygous retina reaches the ora serrata in advance of wild-type (FIGS. 19A and 19B, arrowheads). By P20 the vascular patterning of both Nuc1 homozygous and wild-type appears to be similar with more large caliber vessels present in the Nuc1 homozygotes (FIGS. 19C and 19D). By P120, the Nuc1 homozygote shows clear difference in the morphology and patterning of the vasculature compared with wild-type (FIGS. 19E and F). Evidence of microaneurysm formation in Nuc1 homozygotes (FIG. 19F, small arrows) and intravascular deposits was identified (FIG. 19G, asterisks). Confocal microscopy (FIG. 19H) shows blockage of blood flow inside some vessels (arrowheads).

FIGS. 20A-20F show HUVEC cells cultured in EGM2MV media in accordance with the manufacturer's (Cambrex USA) protocol. When not grown on Matrigel, no tubes are formed (FIGS. 20A and 20B). To determine the optimum cell number that would produce tubes for subsequent studies, different numbers of cells we seeded on Matrigel as shown below. When 15,000 HUVEC cells were cultured (in 96-well plates) on 50 μl of Matrigel, tube formation is evident (arrow in FIG. 20C). If 10,000 HUVEC cells were seeded on 50 μl of Matrigel, thinner tubes were formed (arrow in FIG. 20D). Seeding of 5,000 HUVEC cells on 50 μl of Matrigel, also resulted in tube formation (arrow in FIG. 20E). When 2,500 HUVEC cells on 50 μl of Matrigel were seeded, no tube formation was observed (FIG. 20F). Magnifications: A, B: 20×, C-F: 5×.

FIGS. 21A and B are photomicrographs showing immunolabeling of β-Crystallin expression in HUVEC cells forming tubes on a matrigel substrate. A: Immunofluorescent labeling of β-crystallins in tubes formed from HUVEC cells on Matrigel (arrows). FIG. 21B is the negative control. Magnification: 20×

FIG. 22 is a Western blot showing purified β_{H}-cystallin (Lane 1) and γ-crystallin (Lane 2) as seen by SDS-PAGE. The crystallins were isolated from young bovine lens by gel exclusion chromatography.

FIGS. 23A-23F show the morphology of cultured HUVEC cells exposed to VEGF antibody and β/γ crystallin antibodies. β/γ-crystallin antibodies (1 μl) as well as antibodies to VEGF (1 μl) inhibit the process of tube formation in HUVEC cultures on Matrigel. FIG. 23A demonstrates the extent of tube formation in a representative control culture. FIGS. 23B-23F demonstrate reduced tube-like structure formation in identical cultures supplemented with antibodies to β-crystallins, γ-crystallins and VEGF singly and in combination. Magnification: 5×.

FIG. 24 is a graph showing the effect of antibody addition on HUVEC tube formation by taking average length of all measured tubes (n=7).

FIGS. 25A-25F are photomicrographs of HUVEC cells cultured with purified crystallin and VEGF proteins. HUVEC cells grown in a Matrigel matrix were treated with VEGF (FIG. 25C), β-crystallin (FIG. 25D) and γ crystallin (E) proteins show longer tubes (arrow heads) compared to controls (FIGS. 25A and B).

FIG. 26 is a graph that quantifies the effect of purified crystallin and VEGF proteins on HUVEC cells. HUVEC cells cultured with VEGF, β-crystallin, and γ crystallin proteins show longer tubes (arrow heads) compared to controls (A and B). Comparisons were performed by measuring the length of all the tubes in three different fields, on seven different culture plates indicated reduced length compared to controls. Scale bar 0.5 mm Magnification: 5×

FIG. 27 shows Matrigel-Stimulated Tube Formation in Human Retinal Endothelial Cells (HREC). HREC were cultured in EGM2MV media according to the manufacturer's protocol either in the absence (FIG. 25A), or presence (FIG. 25B) of pre-coated Matrigel matrix (Cambrex). (4× magnification)

FIG. 28 is a graph showing the effect of Exogenous γ-crystallins, VEGF, and PEDF on Tube Length in Cultured Human Retinal Endothelial Cells (HREC): HREC grown on Matrigel-coated tissue culture plates in EGM2MV media with VEGF omitted from the media supplement. Cells were treated with BSA (control), 0.2 μg/ml VEGF, 0.6 μg/ml VEGF, concentrations of β-crystallins ranging from 0.5 μg/ml to 1.0 μg/ml, or 0.2 μg/ml PEDF. Average tube length was calculated after measuring all of the tubes formed in three separate optical fields from 7 individual culture dishes.

FIG. 29 is a Western Blot of 23 day-old Rat retina and SVGA (human astrocyte) cell lysates immunoprecipitated with anti-VEGF (V) and anti-γ crystallin antisera (g) and immunoblotted with anti-γ crystallin antiserum. Lane 1: Total lysate of Nuc-1 −/− retina. Lanes 2&3: Nuc-1 retina lysate immunoprecipitated with anti-γ crystallin (lane 2) and anti-VEGF (lane 3). Lanes 4 & 5: wild type (Sprague Dawley) retinal lysate immunopresipitated with anti-γ crystallin (lane 4) and anti-VEGF (lane 5). Lanes 6 & 7: SVGA human astrocyte cell lysate immunoprecipitated with anti-gamma crystallin (lane 6) and anti-VEGF (lane 7). The arrow at left identifies the γ crystallin band.

### DETAILED DESCRIPTION OF THE INVENTION

The invention features compositions and methods that are useful for modulating angiogenesis, vasculogenesis, blood vessel stabilization, regression, persistence, or remodeling. As reported in more detail below, the invention is based, at least in part, on the discovery that β/γ-crystallins are expressed in the ocular vasculature, where they modulate blood vessel formation and function.

### Crystallins

Crystallins are the most abundant soluble proteins of the lens of the eye. They have specialized roles in the refractive and transparent properties of the lens, but are also expressed in other tissues. Three major families of crystallins, α, β, and γ, are ubiquitously represented in all vertebrates. The function of α-crystallin is best understood. It is a small heat shock protein with anti-aggregative chaperone-like activity. αB-crystallin is inducible by a variety of stresses, including hypoxia (Mayuri et al., FEMS Microbiol Lett. 211(2): 231-7, 2002), and has enhanced expression during oncogenic transformation and in neurodegenerative disorders. It has been shown to play a regulatory role in the nucleus (Bhat et al., Eur J Cell Biol, 78(2): 143-50, 1999). Other reported roles include possible regulatory roles in apoptosis (Liu et al., Exp Eye Res, 79(6): 393-403, 2004) and actin dynamics (Maddala et al., Exp Cell Res, 306(1): 203-15, 2005). The β- and γ-crystallins are evolutionarily and structurally related members of a βγ superfamily, which also includes micro-organism stress proteins and vertebrate proteins associated with cell differentiation, morphological change and cell regulation. The non-lens functions of β and γ-crystallins are less well characterized.

As reported herein, crystallin polypeptides (e.g., β, γ crystallins) were found to function in the ocular vasculature. A rat with a spontaneous mutation (the Nuc1 mutation) has abnormal, eye specific, vascular development (Zhang et al. Dev Dyn. 234(1): 36-47, 2005). Nuc1 rats manifested persistence of the fetal vascular system including the hyaloid vessels even after development of the retinal vessels. Abnormalities in retinal vascular development including altered growth, patterning, remodeling and regression are present in Nuc1 rats. Expression of β and β crystallins in this model were discovered to be spatially and temporally associated with the persistent hyaloid vessels. In normal eyes astrocytes present at the leading edge of developing retinal vessels expressed not only VEGF, but also β and β crystallins. In evaluating the human correlate disease, Persistent Fetal Vasculature (PFV), astrocytes were found to express both VEGF and the β-γ-crystallins. Astrocytes utilized VEGF secretion to induce vessel formation in the vanguard of the developing retina. In an in-vitro model of chemical hypoxia, astrocytes induced to express VEGF, also expressed β- and β-crystallin indicating that β/γ-crystallins affect the differentiation process of cultured human vascular endothelial cells.

### Pathological Neovascularization

Given that crystallins are ubiquitously expressed, the modulation of crystallin expression or biological activity is likely to be broadly useful for the treatment or prevention of diseases or disorders that can be ameliorated by the modulation of angiogenesis, or blood vessel remodeling or stabilization. Diseases and disorders susceptible to treatment by the modulation of crystallin expression or biological activity include those characterized by abnormal, diminished or excess blood vessel formation; examples include, but are not limited to, pathological neovascular disorders, such as ocular neovascularization (iris, retinal choroidal); blood vessels to solid tumors or neoplasia; vascular malformations both benign and malignant; vascular abnormalities in development, such as hemangiomas, vascular malformations or the failure to develop normal structures related to abnormal blood vessel development. Disorders characterized by the absence of vessel formation include birth defects, vascular insufficiency, and failure to develop collateral blood vessels in response to stress, such as ischemia; examples include but are not limited to peripheral vascular or coronary vascular disease) disorders that require an alteration in vascular remodeling, including cancer, arthritis, atherosclerosis, restenosis after angioplasty, systemic and pulmonary hypertension, atherosclerosis, embryonic or fetal development, or vascular response to common or atypical disease. In particular diseases, such as restenosis, the remodeling process involves endothelial cell injury and/or dysfunction that results in intimal/medial thickening). In addition, the invention provides methods and compositions for the treatment of diseases or disorders that require an increase in blood vessel formation (e.g., peripheral artery disease, limb ischemis, cardiac ischemia, stroke. In particular embodiments, the invention is useful for the treatment of pathological ocular neovascularization.

### Pathological Ocular Neovascularization

Neovascularization leads to blindness in a large number of ocular disorders, including but not limited to age-related macular degeneration, diabetic retinopathy, retinopathy of prematurity, and other vascular occlusive diseases such as the occlusive vasculopathies (retinal vein or artery occlusion), sickle cell retinopathy and other, as well as neovascularization resulting from inflammatory stimuli as occurs in uveitis, trauma or other. Congenital or developmental as well as neovascular vascular malformations may benefit from treatment affecting vascular remodeling, regression, stabilization and other. These methods provide for treating or preventing such diseases by reducing or remodelling excess/abnormal blood vessel formation or enhancing stability of normal vessels or for providing regulation of abnormally diminished blood vessel formation.

Diabetic Retinopathy

Diabetic Retinopathy is the most common retinal vascular cause of vision loss. Diabetes causes altered permeability of the retinal microvasculature, resulting in abnormally leaky vessels. The earliest phase of the disease is known as background diabetic retinopathy. In this phase, the arteries in the retina become weakened and leak, forming small, dot-like hemorrhages. These leaking vessels often lead to swelling or edema in the retina and decreased vision. The next stage of pathology is known as proliferative diabetic retinopathy. In this stage, microcirculatory compromise causes areas of the retina to become ischemic. Retinal hypoxia results in neovascularization and vascular leakage. Unfortunately, these delicate vessels hemorrhage easily. Blood may leak into the retina and vitreous and serous components of blood lead to formation of retinal edema. In the later phases of the disease, continued abnormal vessel growth leads to hemorrhage while treatment induced or spontaneous regression of vessels leads to scar tissue formation that may cause serious problems such as retinal detachment and glaucoma.

Macular Degeneration

Macular degeneration is a disorder that affects the macula causing decreased visual acuity and possible loss of central vision. Age-related macular degeneration (AMD) is the leading cause of blindness in people over 50 years of age. Dry macular degeneration is characterized by a slowly progressive retinal degeneration leading to diminished central vision. The more rapidly progressive wet form of the disease may arise at anytime in eyes with dry disease, and is characterized by a proliferation of choroidal vessels that bleed, leak fluid and result in scar formation. Although there are a number of factors that contribute to the development of AMD, the principal cause of rapid vision loss is choroidal neovascularization, the development of abnormal blood vessels that particularly effects the central region of the macula and results in central vision loss.

Retinopathy of Prematurity

Retinopathy of prematurity (ROP) is a potentially blinding eye disorder that primarily affects premature infants that are born before 31 weeks of gestation. It is the leading cause of new blindness in the newborn population. ROP is associated with abnormal retinal neovascularization. The abnormal blood vessels are a response to ischemia and present at the margin of the still developing retina in the premature newborn. The vessels formed in patients with ROP are fragile and can hemorrhage, but the most difficult aspect to treat is the formation of vascular associated scar tissue that pulls the retina out of position (traction retinal detachment) leading to blindness.

The lower the infants birthweight, the more likely that infant is to develop ROP. There are approximately 3.9 million infants born in the U.S. each year, about 14,000-16,000 of these infants are affected by some degree of ROP. About 1,100-1,500 infants annually develop ROP that is severe enough to require medical treatment; and approximately 400-600 infants each year in the US become legally blind for their entire life from ROP. Accordingly, the invention provides methods for treating or preventing ROP pre- or post-natally.

### Therapeutic Methods

The present invention provides methods of treating a vascular disease, disorder or symptom thereof that can be ameliorated by the modulation of angiogenesis, vasculogenesis, blood vessel stabilization, regression, persistence, or remodeling. The methods comprise administering a therapeutically effective amount of a pharmaceutical composition comprising an agent described herein (e.g., an agent that increases or decreases crystallin polypeptide expression or biological activity) to a subject (e.g., a mammal such as a human). Thus, in one embodiment the invention features a method of treating a subject suffering from or susceptible to a disease or disorder or symptom thereof that requires an increase in blood vessel formation or stabilization. Alternatively, the invention provides compositions and methods for reducing pathological neovascularization, particularly pathological ocular neovascularization. The method includes the step of administering to the mammal a therapeutic amount of an agent described herein sufficient to treat the vascular disease or disorder or symptom thereof, under conditions such that the disease or disorder is treated.

The methods herein include administering to the subject (including a subject identified as in need of such treatment) an effective amount of an agent described herein, or a composition described herein to produce such effect. Identifying a subject in need of such treatment can be in the judgment of a subject or a health care professional and can be subjective (e.g. opinion) or objective (e.g. measurable by a test or diagnostic method).

The therapeutic methods of the invention, which include prophylactic treatment, in general comprise administration of a therapeutically effective amount of the agents herein, such as a compound of the formulae herein to a subject (e.g., animal, human) in need thereof, including a mammal, particularly a human. Such treatment will be suitably administered to subjects, particularly humans, suffering from, having, susceptible to, or at risk for a vascular disease, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The compounds herein may be also used in the treatment of any other vascular disorders in which modulation of angiogenesis is required or in which pathological neovascularization, particularly pathological ocular neovascularization may be implicated.

In one embodiment, the invention provides a method of monitoring treatment progress. The method includes the step of determining a level of diagnostic marker (Marker) (e.g., any target delineated herein modulated by a compound herein, a protein or indicator thereof, etc.) or diagnostic measurement (e.g., screen, assay) in a subject suffering from or susceptible to a vascular disorder or retinal degeneration or symptoms thereof, in which the subject has been administered a therapeutic amount of a compound herein sufficient to treat the vascular disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

### Crystallin Polynucleotides

In general, the invention features the use of nucleic acid sequences that encode a crystallin polypeptide or biologically active fragment thereof sufficient to modulate angiogenesis, vasculogenesis, blood vessel remodeling, or blood vessel stabilization. Also included in the methods of the invention are nucleic acid molecules containing at least one strand that hybridizes with a crystallin nucleic acid sequence (e.g., inhibitory nucleic acid molecules that reduce crystallin polypeptide expression, such as a dsRNA, siRNA, shRNA, or antisense oligonucleotides, microRNA, ribozymes, aptamers, monoclonal antibodies or other). An isolated nucleic acid molecule can be manipulated using recombinant DNA techniques well known in the art. Thus, a nucleotide sequence contained in a vector in which 5′ and 3′ restriction sites are known, or for which polymerase chain reaction (PCR) primer sequences have been disclosed, is considered isolated, but a nucleic acid sequence existing in its native state in its natural host is not. An isolated nucleic acid may be substantially purified, but need not be. For example, a nucleic acid molecule that is isolated within a cloning or expression vector may comprise only a tiny percentage of the material in the cell in which it resides. Such a nucleic acid is isolated, however, as the term is used herein, because it can be manipulated using standard techniques known to those of ordinary skill in the art.

### Crystallin Polynucleotide Therapy

Polynucleotide therapy featuring a polynucleotide encoding a crystallin protein, variant, or fragment thereof or encoding an inhibitory nucleic acid molecules that reduce crystallin polypeptide expression (e.g., a dsRNA, siRNA, shRNA, or antisense oligonucleotides, (microRNA, ribozymes, aptamers, monoclonal antibodies or other) are therapeutic approaches for treating a vascular disease or disorder. Such nucleic acid molecules can be delivered to cells of a subject having a vascular disease or disorder, such as a disease that requires an increase in blood vessel formation or stabilization. The nucleic acid molecules must be delivered to the cells of a subject in a form in which they can be taken up so that therapeutically effective levels of a crystallin protein or fragment thereof can be produced.

Transducing viral (e.g., retroviral (lentiviral), adenoviral, and adeno-associated viral, herpes viral) vectors can be used for somatic cell gene therapy, especially because of their high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S. 94:10319, 1997). For example, a polynucleotide encoding an crystallin protein, variant, or a fragment thereof, can be cloned into a retroviral vector and expression can be driven from its endogenous promoter, from the retroviral long terminal repeat, or from a promoter specific for a target cell type of interest. Other viral vectors that can be used include, for example, a vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77 S-83S, 1995). Retroviral vectors are particularly well developed and have been used in clinical settings (Rosenberg et al., N. Engl. J. Med 323:370, 1990; Anderson et al., U.S. Pat. No. 5,399,346). Most preferably, a viral vector is used to administer an crystallin polynucleotide systemically.

Non-viral approaches can also be employed for the introduction of therapeutic to a cell of a patient diagnosed as having a vascular disease or disorder. For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Preferably the nucleic acids are administered in combination with a liposome and protamine. Administration should be sufficient to modulate angiogenesis, vasculogenesis, blood vessel remodeling, or blood vessel stabilization.

Gene transfer can also be achieved using non-viral means involving transfection in vitro. Such methods include the use of calcium phosphate, DEAE dextran, electroporation, and protoplast fusion. Liposomes can also be potentially beneficial for delivery of DNA into a cell. Transplantation of normal genes into the affected tissues of a patient can also be accomplished by transferring a normal nucleic acid into a cultivatable cell type ex vivo (e.g., an autologous or heterologous primary cell or progeny thereof), after which the cell (or its descendants) are injected into a targeted tissue.

cDNA expression for use in polynucleotide therapy methods can be directed from any suitable promoter (e.g., the human cytomegalovirus (CMV), simian virus 40 (SV40), Chicken Beta Actin (CBA) or metallothionein promoters). Promiscuous, ubiquitous or tissue/cell specific promoters are all useful in the methods of the invention. In one embodiment, where expression in an ocular tissue is desired, any promoter sufficient to direct expression in the ocular tissue may be used, including, for example, IRBP, Opsin, RPE65, and Bestrophin The use of such promoters is routine. In other embodiments, promoters encompassed by the present invention are regulated by any appropriate mammalian regulatory element. For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

Another therapeutic approach included in the invention involves administration of a recombinant therapeutic, such as a recombinant crystallin protein, variant, or fragment thereof, either directly to the site of a potential or actual disease-affected tissue or systemically (for example, by any conventional recombinant protein administration technique). The dosage of the administered protein depends on a number of factors, including the size and health of the individual patient. For any particular subject, the specific dosage regimes should be adjusted over time according to the individual need and the professional judgment of the person administering or supervising the administration of the compositions.

In one embodiment, crystallin polypeptides are expressed in vascular cells, such as an endothelial cells, endothelial progenitor cells, pericytes, or astrocytes to achieve a therapeutic benefit but this specifically does not exclude any cell of the cells of the target tissues or of the support tissues as potential treatment targets.

### Crystallin Polypeptide Therapies

As reported herein, crystallin polypeptides have direct effects or effects mediated through relevant pathways on blood vessel formation or remodeling. Accordingly, the invention provides therapeutic methods for the treatment of vascular diseases that feature crystallin polypeptides. In one approach, a crystallin polypeptide is provided directly to a tissue that requires an increase or decrease in angiogenesis, vasculogenesis, blood vessel remodeling, or blood vessel stabilization. Crystallin polypeptides for use in therapeutic methods of the invention are provided by methods known in the art including the purification of a crystallin polypeptide from a biological sample that endogenously produces the polypeptide or the recombinant production of the crystallin polypeptide.

In general, crystallin polypeptides, variants, and fragments thereof are produced by transformation of a suitable host cell with all or part of a polypeptide-encoding nucleic acid molecule or fragment thereof in a suitable expression vehicle. Those skilled in the field of molecular biology will understand that any of a wide variety of expression systems may be used to provide the recombinant protein. The precise host cell used is not critical to the invention. A polypeptide of the invention may be produced in a prokaryotic host (e.g., E. coli) or in a eukaryotic host (e.g., Sacchamyces cerevisiae, insect cells, e.g., Sf21 cells, or mammalian cells, e.g., NIH 3T3, HeLa, or preferably COS cells). Such cells are available from a wide range of sources (e.g., the American Type Culture Collection, Rockland, Md.; also, see, e.g., Ausubel et al., supra). The method of transformation or transfection and the choice of expression vehicle will depend on the host system selected. Transformation and transfection methods are described, e.g., in Ausubel et al. (supra); expression vehicles may be chosen from those provided, e.g., in Cloning Vectors: A Laboratory Manual (P. H. Pouwels et al., 1985, Supp. 1987).

A variety of expression systems exist for the production of the polypeptides of the invention. Expression vectors useful for producing such polypeptides include, without limitation, chromosomal, episomal, and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof.

One particular bacterial expression system for polypeptide production is the E. coli pET expression system (Novagen, Inc., Madison, Wis). According to this expression system, DNA encoding a polypeptide is inserted into a pET vector in an orientation designed to allow expression. Since the gene encoding such a polypeptide is under the control of the T7 regulatory signals, expression of the polypeptide is achieved by inducing the expression of T7 RNA polymerase in the host cell. This is typically achieved using host strains that express T7 RNA polymerase in response to IPTG induction. Once produced, recombinant polypeptide is then isolated according to standard methods known in the art, for example, those described herein.

Another bacterial expression system for polypeptide production is the pGEX expression system (Pharmacia). This system employs a GST gene fusion system that is designed for high-level expression of genes or gene fragments as fusion proteins with rapid purification and recovery of functional gene products. The protein of interest is fused to the carboxyl terminus of the glutathione 5-transferase protein from Schistosoma japonicum and is readily purified from bacterial lysates by affinity chromatography using Glutathione Sepharose 4B. Fusion proteins can be recovered under mild conditions by elution with glutathione. Cleavage of the glutathione S-transferase domain from the fusion protein is facilitated by the presence of recognition sites for site-specific proteases upstream of this domain. For example, proteins expressed in pGEX-2T plasmids may be cleaved with thrombin; those expressed in pGEX-3X may be cleaved with factor Xa.

Once the recombinant polypeptide of the invention is expressed, it is isolated, e.g., using affinity chromatography. In one example, an antibody (e.g., produced as described herein) raised against a polypeptide of the invention may be attached to a column and used to isolate the recombinant polypeptide. Lysis and fractionation of polypeptide-harboring cells prior to affinity chromatography may be performed by standard methods (see, e.g., Ausubel et al., supra).

Once isolated, the recombinant protein can, if desired, be further purified, e.g., by high performance liquid chromatography (see, e.g., Fisher, Laboratory Techniques In Biochemistry and Molecular Biology, eds., Work and Burdon, Elsevier, 1980). Polypeptides of the invention, particularly short peptide fragments, can also be produced by chemical synthesis (e.g., by the methods described in Solid Phase Peptide Synthesis, 2nd ed., 1984 The Pierce Chemical Co., Rockford, Ill.). These general techniques of polypeptide expression and purification can also be used to produce and isolate useful peptide fragments or analogs (described herein).

### Crystallin Polypeptides and Analogs

Also included in the invention are crystallin polypeptides, variants, or fragments thereof containing at least one alteration relative to a reference sequence. Desirably, such variants, fragments and analogs maintain at least one biological function of a full length crystallin polypeptide (i.e., the modulation of angiogenesis, vasculogenesis, blood vessel remodeling, or blood vessel stabilization). Altered crystallin polypeptides include those having certain mutations, deletions, insertions, or post-translational modifications. The invention further includes analogs of any naturally-occurring polypeptide of the invention. Analogs can differ from naturally-occurring polypeptides of the invention by amino acid sequence differences, by post-translational modifications, or by both. Analogs of the invention will generally exhibit at least 85%, more preferably 90%, and most preferably 95% or even 99% identity with all or part of a naturally-occurring amino acid sequence of the invention. The length of sequence comparison is at least 10, 13, 15 amino acid residues, preferably at least 25 amino acid residues, and more preferably more than 35 amino acid residues. Again, in an exemplary approach to determining the degree of identity, a BLAST program may be used, with a probability score between e⁻³ and e⁻¹⁰⁰ indicating a closely related sequence. Modifications include in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally-occurring polypeptides of the invention by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs which contain residues other than L-amino acids, e.g., D-amino acids or non-naturally occurring or synthetic amino acids.

In addition to full-length polypeptides, the invention also includes fragments of any one of the polypeptides of the invention. As used herein, the term “a fragment” means at least 5, 10, 13, or 15 amino acids. In other embodiments a fragment is at least 20 contiguous amino acids, at least 30 contiguous amino acids, or at least 50 contiguous amino acids, and in other embodiments at least 60 to 80 or more contiguous amino acids. Fragments of the invention can be generated by methods known to those skilled in the art or may result from normal protein processing (e.g., removal of amino acids from the nascent polypeptide that are not required for biological activity or removal of amino acids by alternative mRNA splicing or alternative protein processing events).

### Crystallin Antibodies

In another approach, the invention features methods for reducing angiogenesis, vasculogenesis, blood vessel remodeling, or blood vessel stabilization, for example, by reducing the biological activity of a crystallin polypeptides. Methods for reducing the biological activity of a crystallin polypeptide include administering to a subject in need thereof an antibody that specifically binds and disrupts the biological activity of a crystallin polypeptide. The use of such polypeptides is described herein, for example, at Example 15. Antibodies are well known to those of ordinary skill in the science of immunology. As used herein, the term “antibody” means not only intact antibody molecules, but also fragments of antibody molecules that retain immunogen binding ability. Such fragments are also well known in the art and are regularly employed both in vitro and in vivo. Accordingly, as used herein, the term “antibody” means not only intact immunoglobulin molecules but also the well-known active fragments F(ab)₂, and Fab. F(ab′)₂, and Fab fragments which lack the Fc fragment of intact antibody, clear more rapidly from the circulation, and may have less non-specific tissue binding of an intact antibody (Wahl et al., J. Nucl. Med. 24:316-325 (1983). The antibodies of the invention comprise whole native anti-bodies, bispecific antibodies; chimeric antibodies; Fab, Fab′, single chain V region fragments (scFv) and fusion polypeptides.

In one embodiment, an antibody that binds a crystallin polypeptide is monoclonal. Alternatively, the anti-crystallin antibody is a polyclonal antibody. The preparation and use of polyclonal antibodies are also known the skilled artisan. The invention also encompasses hybrid antibodies, in which one pair of heavy and light chains is obtained from a first antibody, while the other pair of heavy and light chains is obtained from a different second antibody. Such hybrids may also be formed using humanized heavy and light chains. Such antibodies are often referred to as “chimeric” antibodies.

In general, intact antibodies are said to contain “Fc” and “Fab” regions. The Fc regions are involved in complement activation and are not involved in antigen binding. An antibody from which the Fc′ region has been enzymatically cleaved, or which has been produced without the Fc′ region, designated an “F(abα)₂” fragment, retains both of the antigen binding sites of the intact antibody. Similarly, an antibody from which the Fc region has been enzymatically cleaved, or which has been produced without the Fc region, designated an “Fab′” fragment, retains one of the antigen binding sites of the intact antibody. Fabα fragments consist of a covalently bound antibody light chain and a portion of the antibody heavy chain, denoted “Fd.” The Fd fragments are the major determinants of antibody specificity (a single Fd fragment may be associated with up to ten different light chains without altering antibody specificity). Isolated Fd fragments retain the ability to specifically bind to immunogenic epitopes.

Antibodies can be made by any of the methods known in the art utilizing crystallin polypeptides, or immunogenic fragments thereof, as an immunogen. One method of obtaining antibodies is to immunize suitable host animals with an immunogen and to follow standard procedures for polyclonal or monoclonal anti-body production. The immunogen will facilitate presentation of the immunogen on the cell surface. Immunization of a suitable host can be carried out in a number of ways. Nucleic acid sequences encoding a crystallin polypeptide, or immunogenic fragments thereof, can be provided to the host in a delivery vehicle that is taken up by immune cells of the host. The cells will in turn express the receptor on the cell surface generating an immunogenic response in the host. Alternatively, nucleic acid sequences encoding a crystallin polypeptide or immunogenic fragments thereof, can be expressed in cells in vitro, followed by isolation of the receptor and administration of the receptor to a suitable host in which antibodies are raised.

Using either approach, antibodies can then be purified from the host. Antibody purification methods may include salt precipitation (for example, with ammonium sulfate), ion exchange chromatography (for example, on a cationic or anionic exchange column preferably run at neutral pH and eluted with step gradients of increasing ionic strength), gel filtration chromatography (including gel filtration HPLC), and chromatography on affinity resins such as protein A, protein G, hydroxyapatite, and anti-immunoglobulin.

Antibodies can be conveniently produced from hybridoma cells engineered to express the antibody. Methods of making hybridomas are well known in the art. The hybridoma cells can be cultured in a suitable medium, and spent medium can be used as an antibody source. Polynucleotides encoding the antibody of interest can in turn be obtained from the hybridoma that produces the antibody, and then the antibody may be produced synthetically or recombinantly from these DNA sequences. For the production of large amounts of antibody, it is generally more convenient to obtain an ascites fluid. The method of raising ascites generally comprises injecting hybridoma cells into an immunologically naive histocompatible or immunotolerant mammal, especially a mouse. The mammal may be primed for ascites production by prior administration of a suitable composition; e.g., Pristane.

Monoclonal antibodies (Mabs) produced by methods of the invention can be “humanized” by methods known in the art. “Humanized” antibodies are antibodies in which at least part of the sequence has been altered from its initial form to render it more like human immunoglobulins. Techniques to humanize antibodies are particularly useful when non-human animal (e.g., murine) antibodies are generated. Examples of methods for humanizing a murine antibody are provided in U.S. Pat. Nos. 4,816,567, 5,530,101, 5,225,539, 5,585,089, 5,693,762 and 5,859,205.

### Inhibitory Nucleic Acids

Inhibitory nucleic acid molecules are those oligonucleotides that inhibit the expression or activity of a crystallin polypeptide. Such oligonucleotides include single and double stranded nucleic acid molecules (e.g., DNA, RNA, and analogs thereof) that bind a nucleic acid molecule that encodes a crystallin polypeptide (e.g., antisense molecules, siRNA, shRNA, microRNA) as well as nucleic acid molecules that bind directly to a crystallin polypeptide to modulate its biological activity (e.g., aptamers).

Ribozymes

Catalytic RNA molecules or ribozymes that include an antisense crystallin sequence of the present invention can be used to inhibit expression of a crystallin nucleic acid molecule in vivo. The inclusion of ribozyme sequences within antisense RNAs confers RNA-cleaving activity upon them, thereby increasing the activity of the constructs. The design and use of target RNA-specific ribozymes is described in Haseloff et al., Nature 334:585-591. 1988, and U.S. Patent Application Publication No. 2003/0003469 A1, each of which is incorporated by reference.

Accordingly, the invention also features a catalytic RNA molecule that includes, in the binding arm, an antisense RNA having between eight and nineteen consecutive nucleobases. In preferred embodiments of this invention, the catalytic nucleic acid molecule is formed in a hammerhead or hairpin motif. Examples of such hammerhead motifs are described by Rossi et al., Aids Research and Human Retroviruses, 8:183, 1992. Example of hairpin motifs are described by Hampel et al., “RNA Catalyst for Cleaving Specific RNA Sequences,” filed Sep. 20, 1989, which is a continuation-in-part of U.S. Ser. No. 07/247,100 filed Sep. 20, 1988, Hampel and Tritz, Biochemistry, 28:4929, 1989, and Hampel et al., Nucleic Acids Research, 18: 299, 1990. These specific motifs are not limiting in the invention and those skilled in the art will recognize that all that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule.

Small hairpin RNAs consist of a stem-loop structure with optional 3′ UU-overhangs. While there may be variation, stems can range from 21 to 31 bp (desirably 25 to 29 bp), and the loops can range from 4 to 30 bp (desirably 4 to 23 bp). For expression of shRNAs within cells, plasmid vectors containing either the polymerase III H1-RNA or U6 promoter, a cloning site for the stem-looped RNA insert, and a 4-5-thymidine transcription termination signal can be employed. The Polymerase III promoters generally have well-defined initiation and stop sites and their transcripts lack poly(A) tails. The termination signal for these promoters is defined by the polythymidine tract, and the transcript is typically cleaved after the second uridine. Cleavage at this position generates a 3′ UU overhang in the expressed shRNA, which is similar to the 3′ overhangs of synthetic siRNAs. Additional methods for expressing the shRNA in mammalian cells are described in the references cited above.

siRNA

Short twenty-one to twenty-five nucleotide double-stranded RNAs are effective at down-regulating gene expression (Zamore et al., Cell 101: 25-33; Elbashir et al., Nature 411: 494-498, 2001, hereby incorporated by reference). The therapeutic effectiveness of an siRNA approach in mammals was demonstrated in vivo by McCaffrey et al. (Nature 418: 38-39. 2002).

Given the sequence of a target gene, siRNAs may be designed to inactivate that gene. Such siRNAs, for example, could be administered directly to an affected tissue, or administered systemically. The nucleic acid sequence of an crystallin gene can be used to design small interfering RNAs (siRNAs). The 21 to 25 nucleotide siRNAs may be used, for example, as therapeutics to treat a vascular disease or disorder.

The inhibitory nucleic acid molecules of the present invention may be employed as double-stranded RNAs for RNA interference (RNAi)-mediated knock-down of crystallin expression. In one embodiment, crystallin expression is reduced in an endothelial cell or an astrocyte. RNAi is a method for decreasing the cellular expression of specific proteins of interest (reviewed in Tuschl, Chembiochem 2:239-245, 2001; Sharp, Genes & Devel. 15:485-490, 2000; Hutvagner and Zamore, Curr. Opin. Genet Devel. 12:225-232, 2002; and Hannon, Nature 418:244-251, 2002). The introduction of siRNAs into cells either by transfection of dsRNAs or through expression of siRNAs using a plasmid-based expression system is increasingly being used to create loss-of-function phenotypes in mammalian cells.

In one embodiment of the invention, doublestranded RNA (dsRNA) molecule is made that includes between eight and nineteen consecutive nucleobases of a nucleobase oligomer of the invention. The dsRNA can be two distinct strands of RNA that have duplexed, or a single RNA strand that has self-duplexed (small hairpin (sh)RNA). Typically, dsRNAs are about 21 or 22 base pairs, but may be shorter or longer (up to about 29 nucleobases) if desired. dsRNA can be made using standard techniques (e.g., chemical synthesis or in vitro transcription). Kits are available, for example, from Ambion (Austin, Tex.) and Epicentre (Madison, Wis.). Methods for expressing dsRNA in mammalian cells are described in Brummelkamp et al. Science 296:550-553, 2002; Paddison et al. Genes & Devel. 16:948-958, 2002. Paul et al. Nature Biotechnol. 20:505-508, 2002; Sui et al. Proc. Natl. Acad. Sci. USA 99:5515-5520, 2002; Yu et al. Proc. Natl. Acad. Sci. USA 99:6047-6052, 2002; Miyagishi et al. Nature Biotechnol. 20:497-500, 2002; and Lee et al. Nature Biotechnol. 20:500-505 2002, each of which is hereby incorporated by reference.

shRNAs

Small hairpin RNAs consist of a stem-loop structure with optional 3′ UU-overhangs. While there may be variation, stems can range from 21 to 31 bp (desirably 25 to 29 bp), and the loops can range from 4 to 30 bp (desirably 4 to 23 bp). For expression of shRNAs within cells, plasmid vectors containing either the polymerase III H1-RNA or U6 promoter, a cloning site for the stem-looped RNA insert, and a 4-5-thymidine transcription termination signal can be employed. The Polymerase III promoters generally have well-defined initiation and stop sites and their transcripts lack poly(A) tails. The termination signal for these promoters is defined by the polythymidine tract, and the transcript is typically cleaved after the second uridine. Cleavage at this position generates a 3′ UU overhang in the expressed shRNA, which is similar to the 3′ overhangs of synthetic siRNAs. Additional methods for expressing the shRNA in mammalian cells are described in the references cited above.

microRNAs

microRNAs (miRNAs) are an abundant class of endogenous non-protein-coding small RNAs, which negatively regulate gene expression at the posttrascriptional level in many developmental and metabolic processes. miRNAs regulate a variety of biological processes, including developmental timing, signal transduction, tissue differentiation and maintenance, disease, and carcinogenesis. MicroRNAs represent a means to down regulate crystallin expression.

### Aptamers

Nucleic acid aptamers are single-stranded nucleic acid (DNA or RNA) ligands that function by folding into a specific globular structure that dictates binding to target proteins or other molecules with high affinity and specificity, as described by Osborne et al., Curr. Opin. Chem. Biol. 1:5-9, 1997; and Cerchia et al., FEBS Letters 528:12-16, 2002. Desirably, the aptamers are small, approximately ˜15 KD. The aptamers are isolated from libraries consisting of some 10¹⁴-10¹⁵ random oligonucleotide sequences by a procedure termed SELEX (systematic evolution of ligands by exponential enrichment). See Tuerk et al., Science, 249:505-510, 1990; Green et al., Methods Enzymology. 75-86, 1991; Gold et al., Annu. Rev. Biochem., 64: 763-797, 1995; Uphoff et al., Curr. Opin. Struct. Biol., 6: 281-288, 1996. Methods of generating aptamers are known in the art and are described, for example, in U.S. Pat. Nos. 6,344,318, 6,331,398, 6,110,900, 5,817,785, 5,756,291, 5,696,249, 5,670,637, 5,637,461, 5,595,877, 5,527,894, 5,496,938, 5,475,096, 5,270,163, and in U.S. Patent Application Publication Nos. 20040241731, 20030198989, 20030157487, and 20020172962.

An aptamer of the invention is capable of binding with specificity to a crystallin polypeptide expressed by a cell of interest. “Binding with specificity” means that non-crystallin polypeptides are either not specifically bound by the aptamer or are only poorly bound by the aptamer. In general, aptamers typically have binding constants in the picomolar range. Particularly useful in the methods of the invention are aptamers having apparent dissociation constants of 1, 10, 15, 25, 50, 75, or 100 nM. Because many cells of interest express one or more crystallin polypeptides (e.g., β, γ crystallin), in one embodiment, the invention features a pharmaceutical composition that contains two or more aptamers, each of which recognizes a different crystallin polypeptide.

In one embodiment, γ- or β-crystallin is the molecular target of the aptamer. Because aptamers can act as direct antagonists of the biological function of proteins, aptamers that target a crystallin polypeptide can be used to modulate angiogenesis, vasculogenesis, blood vessel stabilization or remodeling. The therapeutic benefit of such aptamers derives primarily from the biological antagonism caused by aptamer binding.

The invention encompasses stabilized aptamers having modifications that protect against 3′ and 5′ exonucleases as well as endonucleases. Such modifications desirably maintain target affinity while increasing aptamer stability in vivo. In various embodiments, aptamers of the invention include chemical substitutions at the ribose and/or phosphate and/or base positions of a given nucleobase sequence. For example, aptamers of the invention include chemical modifications at the 2′ position of the ribose moiety, circularization of the aptamer, 3′ capping and ‘spiegelmer’ technology. Such modifications are known in the art and are described herein. Aptamers having A and G nucleotides sequentially replaced with their 2′-OCH3 modified counterparts are particularly useful in the methods of the invention. Such modifications are typically well tolerated in terms of retaining aptamer affinity and specificity. In various embodiments, aptamers include at least 10%, 25%, 50%, or 75% modified nucleotides. In other embodiments, as many as 80-90% of the aptatmer's nucleotides contain stabilizing substitutions. In other embodiments, 2′-OMe aptamers are synthesized. Such aptamers are desirable because they are inexpensive to synthesize and natural polymerases do not accept 2′-OMe nucleotide triphosphates as substrates so that 2′-OMe nucleotides cannot be recycled into host DNA. A fully 2′-O-methyl aptamer, named ARC245, was reported to be so stable that degradation could not be detected after 96 hours in plasma at 37° C. or after autoclaving at 125° C. Using methods, described herein, aptamers will be selected for reduced size and increased stability. In one embodiment, aptamers having 2′-F and 2′-OCH₃ modifications are used to generate nuclease resistant aptamers. Other modifications that stabilize aptamers are known in the art and are described, for example, in U.S. Pat. No. 5,580,737; and in U.S. Patent Application Publication Nos. 20050037394, 20040253679, 20040197804, and 20040180360.

Using standard methods crystallin-specific aptamers can be selected that bind virtually any crystallin polypeptide known in the art. Exemplary aptamers useful for targeting an angiogenic cell type include EYE0001, and those that target angiopoietin-2 (White et al., Proc Natl Acad Sci USA. 2003 Apr. 29; 100(9):5028-33 and pigpen (Blank et al., J Biol. Chem. 2001 May 11; 276(19):16464-8).

### Delivery of Nucleobase Oligomers

Naked inhibitory nucleic acid molecules, or analogs thereof, are capable of entering mammalian cells and inhibiting expression of a gene of interest. Nonetheless, it may be desirable to utilize a formulation that aids in the delivery of oligonucleotides or other nucleobase oligomers to cells (see, e.g., U.S. Pat. Nos. 5,656,611, 5,753,613, 5,785,992, 6,120,798, 6,221,959, 6,346,613, and 6,353,055, each of which is hereby incorporated by reference).

### Pharmaceutical Compositions

The present invention contemplates pharmaceutical preparations comprising a crystallin protein, a polynucleotide that encodes a crystallin protein, an aptamer that binds a crystallin polypeptide, or a crystallin inhibitory nucleic acid molecule (e.g., a polynucleotide that hybridizes to and interferes with the expression of an crystallin polynucleotide), together with a pharmaceutically acceptable carrier. Polynucleotides of the invention may be administered as part of a pharmaceutical composition. The compositions should be sterile and contain a therapeutically effective amount of the polypeptides or nucleic acid molecules in a unit of weight or volume suitable for administration to a subject.

These compositions ordinarily will be stored in unit or multi-dose containers, for example, sealed ampoules or vials, as an aqueous solution or as a lyophilized formulation for reconstitution. As an example of a lyophilized formulation, 10 mL vials are filled with 5 mL of sterile-filtered 1% (w/v) aqueous crystallin polynucleotide solution, such as an aqueous solution of crystallin polynucleotide or polypeptide, and the resulting mixture can then be lyophilized. The infusion solution can be prepared by reconstituting the lyophilized material using sterile Water-for-Injection (WFI).

The crystallin polynucleotide, or polypeptide, or analogs may be combined, optionally, with a pharmaceutically acceptable excipient. The term “pharmaceutically-acceptable excipient” as used herein means one or more compatible solid or liquid filler, diluents or encapsulating substances that are suitable for administration into a human. The term “carrier” denotes an organic or inorganic ingredient, natural or synthetic, with which the active ingredient is combined to facilitate administration. The components of the pharmaceutical compositions also are capable of being co-mingled with the molecules of the present invention, and with each other, in a manner such that there is no interaction that would substantially impair the desired pharmaceutical efficacy.

The compositions can be administered in effective amounts. The effective amount will depend upon the mode of administration, the particular condition being treated and the desired outcome. It may also depend upon the stage of the condition, the age and physical condition of the subject, the nature of concurrent therapy, if any, and like factors well known to the medical practitioner. For therapeutic applications, it is that amount sufficient to achieve a medically desirable result.

With respect to a subject having an neoplastic disease or disorder, an effective amount is sufficient to stabilize, slow, or reduce the proliferation of the neoplasm. Generally, doses of active polynucleotide compositions of the present invention would be from about 0.01 mg/kg per day to about 1000 mg/kg per day. It is expected that doses ranging from about 50 to about 2000 mg/kg will be suitable. Lower doses will result from certain forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of the crystallin polynucleotide or polypeptide compositions of the present invention.

A variety of administration routes are available. The methods of the invention, generally speaking, may be practiced using any mode of administration that is medically acceptable, meaning any mode that produces effective levels of the active compounds without causing clinically unacceptable adverse effects. Other modes of administration include oral, rectal, topical, intraocular, buccal, intravaginal, intracisternal, intracerebroventricular, intratracheal, nasal, transdermal, within/on implants, e.g., fibers such as collagen, osmotic pumps, or grafts comprising appropriately transformed cells, etc., or parenteral routes. A particular method of administration involves coating, embedding or derivatizing fibers, such as collagen fibers, protein polymers, etc. with therapeutic proteins. Other useful approaches are described in Otto, D. et al., J. Neurosci. Res. 22: 83-91 and in Otto, D. and Unsicker, K. J. Neurosci. 10: 1912-1921.

### Methods of Ocular Delivery

The compositions of the invention are particularly suitable for treating diseases and disorders that require the modulation of blood vessel formation, stabilization (e.g., regression or persistence), or remodelling in the eye. In one embodiment, the invention provides therapeutics for the treatment of diseases, disorders or tissue damage that requires an increase in angiogenesis. In another embodiment, the invention provides therapeutics for the treatment of pathological neovascularization (e.g., pathological ocular neovascularization) or vascular instability resulting in leakage.

In one approach, the compositions of the invention are administered through an ocular device suitable for direct implantation into the vitreous of the eye. The compositions of the invention may be provided in sustained release compositions, such as those described in, for example, U.S. Pat. Nos. 5,672,659 and 5,595,760. Such devices are found to provide sustained controlled release of various compositions to treat the eye without risk of detrimental local and systemic side effects. An object of the present ocular method of delivery is to maximize the amount of drug contained in an intraocular device or implant while minimizing its size in order to prolong the duration of the implant. See, e.g., U.S. Pat. Nos. 5,378,475; 6,375,972, and 6,756,058 and U.S. Publications 20050096290 and 200501269448. Such implants may be biodegradable and/or biocompatible implants, or may be non-biodegradable implants. Biodegradable ocular implants are described, for example, in U.S. Patent Publication No. 20050048099. The implants may be permeable or impermeable to the active agent, and may be inserted into a chamber of the eye, such as the anterior or posterior chambers or may be implanted in or on the sclera, transchoroidal space, or an avascularized region exterior to the vitreous. Alternatively, a contact lens that acts as a depot for compositions of the invention may also be used for drug delivery.

In a preferred embodiment, the implant may be positioned over an avascular region, such as on the sclera, so as to allow for transcleral diffusion of the drug to the desired site of treatment, e.g. the intraocular space and macula of the eye. Furthermore, the site of transcleral diffusion is preferably in proximity to the macula. Examples of implants for delivery of an a composition include, but are not limited to, the devices described in U.S. Pat. Nos. 3,416,530; 3,828,777; 4,014,335; 4,300,557; 4,327,725; 4,853,224; 4,946,450; 4,997,652; 5,147,647; 5,164,188; 5,178,635; 5,300,114; 5,322,691; 5,403,901; 5,443,505; 5,466,466; 5,476,511; 5,516,522; 5,632,984; 5,679,666; 5,710,165; 5,725,493; 5,743,274; 5,766,242; 5,766,619; 5,770,592; 5,773,019; 5,824,072; 5,824,073; 5,830,173; 5,836,935; 5,869,079, 5,902,598; 5,904,144; 5,916,584; 6,001,386; 6,074,661; 6,110,485; 6,126,687; 6,146,366; 6,251,090; and 6,299,895, and in WO 01/30323 and WO 01/28474, all of which are incorporated herein by reference.

Examples include, but are not limited to the following: a sustained release drug delivery system comprising an inner reservoir comprising an effective amount of an agent effective in obtaining a desired local or systemic physiological or pharmacological effect, an inner tube impermeable to the passage of the agent, the inner tube having first and second ends and covering at least a portion of the inner reservoir, the inner tube sized and formed of a material so that the inner tube is capable of supporting its own weight, an impermeable member positioned at the inner tube first end, the impermeable member preventing passage of the agent out of the reservoir through the inner tube first end, and a permeable member positioned at the inner tube second end, the permeable member allowing diffusion of the agent out of the reservoir through the inner tube second end; a method for administering a compound of the invention to a segment of an eye, the method comprising the step of implanting a sustained release device to deliver the compound of the invention to the vitreous of the eye or an implantable, sustained release device for administering a compound of the invention to a segment of an eye; a sustained release drug delivery device comprising: a) a drug core comprising a therapeutically effective amount of at least one first agent effective in obtaining a diagnostic effect or effective in obtaining a desired local or systemic physiological or pharmacological effect; b) at least one unitary cup essentially impermeable to the passage of the agent that surrounds and defines an internal compartment to accept the drug core, the unitary cup comprising an open top end with at least one recessed groove around at least some portion of the open top end of the unitary cup; c) a permeable plug which is permeable to the passage of the agent, the permeable plug is positioned at the open top end of the unitary cup wherein the groove interacts with the permeable plug holding it in position and closing the open top end, the permeable plug allowing passage of the agent out of the drug core, through the permeable plug, and out the open top end of the unitary cup; and d) at least one second agent effective in obtaining a diagnostic effect or effective in obtaining a desired local or systemic physiological or pharmacological effect; or a sustained release drug delivery device comprising: an inner core comprising an effective amount of an agent having a desired solubility and a polymer coating layer, the polymer layer being permeable to the agent, wherein the polymer coating layer completely covers the inner core.

Other approaches for ocular delivery include the use of liposomes to target a compound of the present invention to the eye. For example, the compound may be complexed with liposomes in the manner described above, and this compound/liposome complex injected into patients with an neovascular ocular pathology, using intravenous injection to direct the compound to the desired ocular tissue or cell. Directly injecting the liposome complex into the proximity of the retinal pigment epithelial cells or Bruch's membrane can also provide for targeting of the composition with some forms of neovascular ocular pathology. Further targeting may result from the local release of the agent by light, thermal, laser or other localized liposome release mechanism (similar to Ran Zeimer). In a specific embodiment, the compound is administered via intra-ocular sustained delivery (such as VITRASERT or ENVISION). In a specific embodiment, the compound is delivered by posterior subtenons injection. In another specific embodiment, microemulsion particles containing the compositions of the invention are delivered to ocular tissue to take up lipid from Bruch's membrane, retinal pigment epithelial cells, or both.

Nanoparticles are a colloidal carrier system that has been shown to improve the efficacy of the encapsulated drug by prolonging the serum half-life. Polyalkylcyanoacrylates (PACAs) nanoparticles are a polymer colloidal drug delivery system that is in clinical development, as described by Stella et al., J. Pharm. Sci., 2000. 89: p. 1452-1464; Brigger et al., Int. J. Pharm., 2001.214: p. 3742; Calvo et al., Pharm. Res., 2001. 18: p. 1157-1166; and Li et al., Biol. Pharm. Bull., 2001. 24: p. 662-665. Biodegradable poly (hydroxyl acids), such as the copolymers of poly(acetic acid) (PLA) and poly (lactic-o-glycolide) (PLGA) are being extensively used in biomedical applications and have received FDA approval for certain clinical applications. In addition, PEG-PLGA nanoparticles have many desirable carrier features including (i) that the agent to be encapsulated comprises a reasonably high weight fraction (loading) of the total carrier system; (ii) that the amount of agent used in the first step of the encapsulation process is incorporated into the final carrier (entrapment efficiency) at a reasonably high level; (iii) that the carrier have the ability to be freeze-dried and reconstituted in solution without aggregation; (iv) that the carrier be biodegradable; (v) that the carrier system be of small size; and (vi) that the carrier enhance the particles persistence.

Nanoparticles are synthesized using virtually any biodegradable shell known in the art. In one embodiment, a polymer, such as poly (lactic-acid) (PLA) orpoly (lactic-co-glycolic acid) (PLGA) is used. Such polymers are biocompatible and biodegradable, and are subject to modifications that desirably increase the photochemical efficacy and circulation lifetime of the nanoparticle. In one embodiment, the polymer is modified with a terminal carboxylic acid group (COOH) that increases the negative charge of the particle and thus limits the interaction with negatively charge nucleic acid aptamers. Nanoparticles are also modified with polyethylene glycol (PEG), which also increases the half-life and stability of the particles in circulation. Alternatively, the COOH group is converted to an N-hydroxysuccinimide (NHS) ester for covalent conjugation to amine-modified aptamers.

Biocompatible polymers useful in the composition and methods of the invention include, but are not limited to, polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetage phthalate, carboxylethyl cellulose, cellulose triacetate, cellulose sulphate sodium salt, poly(methyl methacrylate), poly(ethylmethacrylate), poly(butylmethacrylate), poly(isobutylmethacrylate), poly(hexlmethacrylate), poly(isodecylmethacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene poly(ethylene glycol), poly(ethylene oxide), poly(ethylene terephthalate), poly(vinyl alcohols), poly(vinyl acetate, poly vinyl chloride polystyrene, polyvinylpryrrolidone, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutylmethacrylate), poly(hexlmethacrylate), poly(isodecl methacrylate), poly(lauryl methacrylate), poly(phenyl methacrylate), poly(methyl acrylate), poly(isopropyl acrylate), poly(isobutyl acrylate), poly(octadecl acrylate) and combinations of any of these. In one embodiment, the nanoparticles of the invention include PEG-PLGA polymers.

Compositions of the invention may also be delivered topically. For topical delivery, the compositions are provided in any pharmaceutically acceptable excipient that is approved for ocular delivery. Preferably, the composition is delivered in drop form to the surface of the eye. For some application, the delivery of the composition relies on the diffusion of the compounds through the cornea to the interior of the eye.

Those of skill in the art will recognize that the best-treatment regimens for using compounds of the present invention to treat a disease characterized by pathological ocular neovascularization can be straightforwardly determined. This is not a question of experimentation, but rather one of optimization, which is routinely conducted in the medical arts. In vivo studies in nude mice often provide a starting point from which to begin to optimize the dosage and delivery regimes. The frequency of injection will initially be once a week, as has been done in some mice studies. However, this frequency might be optimally adjusted from one day to every two weeks to monthly, depending upon the results obtained from the initial clinical trials and the needs of a particular patient.

Human dosage amounts can initially be determined by extrapolating from the amount of compound used in mice, as a skilled artisan recognizes it is routine in the art to modify the dosage for humans compared to animal models. In certain embodiments it is envisioned that the dosage may vary from between about 1 mg compound/Kg body weight to about 5000 mg compound/Kg body weight; or from about 5 mg/Kg body weight to about 4000 mg/Kg body weight or from about 10 mg/Kg body weight to about 3000 mg/Kg body weight; or from about 50 mg/Kg body weight to about 2000 mg/Kg body weight; or from about 100 mg/Kg body weight to about 1000 mg/Kg body weight; or from about 150 mg/Kg body weight to about 500 mg/Kg body weight. In other embodiments this dose may be about 1, 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, 5000 mg/Kg body weight. In other embodiments, it is envisaged that higher does may be used, such doses may be in the range of about 5 mg compound/Kg body to about 20 mg compound/Kg body. In other embodiments the doses may be about 8, 10, 12, 14, 16 or 18 mg/Kg body weight. Where a composition of the invention is used dosages of 1 mg, 2 mg, 3 mg, 5 mg, 7 mg, 10 mg, 15 mg, 20 mg, or 25 mg can be used per day. Of course, this dosage amount may be adjusted upward or downward, as is routinely done in such treatment protocols, depending on the results of the initial clinical trials and the needs of a particular patient. In various embodiments, compositions of the invention are administered directly to a tissue or organ of interest by direct injection of a protein or inhibitory nucleic acid molecule described herein or by injection of a vector, such as a viral vector encoding a protein or inhibitory nucleic acid molecule of interest. In one approach, a therapeutic composition is administered in or near the target tissue. For example, where the target tissue is an ocular tissue administration is by intraocular or periocular injection.

### Screening Assays

As reported herein, the expression of a crystallin polypeptide is increased in Nuc1 tissues where fetal vasculature fails to regress. Accordingly, compounds that modulate the expression or activity of a crystallin polypeptide, variant, or fragment thereof are useful in the methods of the invention for the treatment or prevention of a disease or disorder that requires modulation of angiogenesis, vasculogenesis, blood vessel stabilization or remodeling. Any number of methods are available for carrying out screening assays to identify such compounds. In one approach, candidate compounds are identified that specifically bind to and alter the activity of a polypeptide of the invention (e.g., a crystallin activity associated with angiogenesis, vasculogenesis, blood vessel stabilization or remodeling). Methods of assaying such biological activities are known in the art and are described herein. The efficacy of such a candidate compound is dependent upon its ability to interact with an crystallin polypeptide, variant, or fragment. Such an interaction can be readily assayed using any number of standard binding techniques and functional assays (e.g., those described in Ausubel et al., supra). For example, a candidate compound may be tested in vitro for interaction and binding with a polypeptide of the invention and its ability to modulate angiogenesis, vasculogenesis, blood vessel stabilization or remodeling. Crystallin's function in angiogenesis, vasculogenesis, blood vessel stabilization or remodeling can be assayed by detecting, for example, tube formation or extension in an endothelial cell where endogenous crystallin expression or activity is perturbed or reduced. Standard methods for perturbing or reducing crystallin expression include mutating or deleting an endogenous crystallin sequence, interfering with crystallin expression using RNAi, or microinjecting a crystallin-expressing cell with an antibody or aptamer that binds crystallin and interferes with its function. Alternatively, angiogenesis, vasculogenesis, blood vessel stabilization or remodeling can be assayed in vivo, for example, in a mouse model in which crystallin has been knocked out by homologous recombination, or any other standard method.

Potential agonists and antagonists of an crystallin polypeptide include organic molecules, peptides, peptide mimetics, polypeptides, nucleic acid molecules (e.g., double-stranded RNAs, siRNAs, antisense polynucleotides, aptamers), and antibodies that bind to a nucleic acid sequence or polypeptide of the invention and thereby inhibit or extinguish its activity. Potential antagonists also include small molecules that bind to the crystallin polypeptide thereby preventing binding to cellular molecules with which the crystallin polypeptide normally interacts (e.g., VEGF), such that the normal biological activity of the crystallin polypeptide is reduced or inhibited. Small molecules of the invention preferably have a molecular weight below 2,000 daltons, more preferably between 300 and 1,000 daltons, and most preferably between 400 and 700 daltons. It is preferred that these small molecules are organic molecules.

In one particular example, a candidate compound that binds to a crystallin polypeptide, variant, or fragment thereof may be identified using a chromatography-based technique. For example, a recombinant polypeptide of the invention may be purified by standard techniques from cells engineered to express the polypeptide (e.g., those described above) and may be immobilized on a column. A solution of candidate compounds is then passed through the column, and a compound specific for the crystallin polypeptide is identified on the basis of its ability to bind to the crystallin polypeptide and be immobilized on the column. To isolate the compound, the column is washed to remove non-specifically bound molecules, and the compound of interest is then released from the column and collected.

Similar methods may be used to isolate a compound bound to a polypeptide microarray. Compounds isolated by this method (or any other appropriate method) may, if desired, be further purified (e.g., by high performance liquid chromatography). In addition, these candidate compounds may be tested for their ability to alter the biological activity of a crystallin polypeptide.

Compounds that are identified as binding to a polypeptide of the invention with an affinity constant less than or equal to 10 mM are considered particularly useful in the invention. Alternatively, any in vivo protein interaction detection system, for example, any two-hybrid assay may be utilized to identify compounds that interact with a crystallin polypeptide. Interacting compounds isolated by this method (or any other appropriate method) may, if desired, be further purified (e.g., by high performance liquid chromatography). Compounds isolated by any approach described herein may be used as therapeutics to treat a vascular disease in a human patient.

In addition, compounds that inhibit the expression of a crystallin nucleic acid molecule whose expression is altered in a patient having a vascular disease or disorder are also useful in the methods of the invention. Any number of methods are available for carrying out screening assays to identify new candidate compounds that alter the expression of a crystallin nucleic acid molecule. In one working example, candidate compounds are added at varying concentrations to the culture medium of cultured cells expressing one of the nucleic acid sequences of the invention. Gene expression is then measured, for example, by microarray analysis, Northern blot analysis (Ausubel et al., supra), or RT-PCR, using any appropriate fragment prepared from the nucleic acid molecule as a hybridization probe. The level of gene expression in the presence of the candidate compound is compared to the level measured in a control culture medium lacking the candidate molecule. A compound that promotes an alteration in the expression of an crystallin gene, or a functional equivalent thereof, is considered useful in the invention; such a molecule may be used, for example, as a therapeutic to treat a vascular disease or disorder in a human patient.

In another approach, the effect of candidate compounds is measured at the level of polypeptide production to identify those that promote an alteration in a crystallin polypeptide level. The level of crystallin polypeptide can be assayed using any standard method. Standard immunological techniques include Western blotting or immunoprecipitation with an antibody specific for an crystallin polypeptide. For example, immunoassays may be used to detect or monitor the expression of at least one of the polypeptides of the invention in an organism. Polyclonal or monoclonal antibodies (produced as described above) that are capable of binding to such a polypeptide may be used in any standard immunoassay format (e.g., ELISA, Western blot, or RIA assay) to measure the level of the polypeptide. In some embodiments, a compound that promotes a decrease in the expression or biological activity of the polypeptide is considered particularly useful. Again, such a molecule may be used, for example, as a therapeutic to delay, ameliorate, or treat a vascular disease in a human patient.

In another embodiment, a nucleic acid described herein is expressed as a transcriptional or translational fusion with a detectable reporter, and expressed in an isolated cell (e.g., mammalian or insect cell) under the control of a heterologous promoter, such as an inducible promoter. The cell expressing the fusion protein is then contacted with a candidate compound, and the expression of the detectable reporter in that cell is compared to the expression of the detectable reporter in an untreated control cell. A candidate compound that alters the expression of the detectable reporter is a compound that is useful for the treatment of vascular disease. In one embodiment, the compound decreases the expression of the reporter.

Each of the DNA sequences listed herein may also be used in the discovery and development of a therapeutic compound for the treatment of vascular disease. The encoded protein, upon expression, can be used as a target for the screening of drugs. Additionally, the DNA sequences encoding the amino terminal regions of the encoded protein or Shine-Delgarno or other translation facilitating sequences of the respective mRNA can be used to construct sequences that promote the expression of the coding sequence of interest. Such sequences may be isolated by standard techniques (Ausubel et al., supra).

The invention also includes novel compounds identified by the above-described screening assays. Optionally, such compounds are characterized in one or more appropriate animal models to determine the efficacy of the compound for the treatment of a vascular disease. Desirably, characterization in an animal model can also be used to determine the toxicity, side effects, or mechanism of action of treatment with such a compound. Furthermore, novel compounds identified in any of the above-described screening assays may be used for the treatment of a vascular disease in a subject. Such compounds are useful alone or in combination with other conventional therapies known in the art.

(Crystallins, may serve as markers of disease with over or under expression being present. In one example Mu crystallins serve as a marker of e.g. retinal degeneration based on the amount present.)

### Test Compounds and Extracts

In general, compounds capable of inhibiting the growth or proliferation of a vascular disease by altering the expression or biological activity of a crystallin polypeptide, variant, or fragment thereof are identified from large libraries of either natural product or synthetic (or semi-synthetic) extracts or chemical libraries according to methods known in the art. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmalMar, U.S. (Cambridge, Mass.).

In one embodiment, test compounds of the invention are present in any combinatorial library known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann, R. N. et al., J. Med. Chem. 37:2678-85, 1994); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, Anticancer. DrugDes. 12:145, 1997).

Examples of methods for the synthesis of molecular libraries can be found in the art, for example in: DeWitt et al., Proc. Natl. Acad. Sci. U.S.A. 90:6909, 1993; Erb et al., Proc. Natl. Acad. Sci. USA 91:11422, 1994; Zuckermann et al., J. Med. Chem. 37:2678, 1994; Cho et al., Science 261:1303, 1993; Carrell et al., Angew. Chem. Int. Ed. Engl. 33:2059, 1994; Carell et al., Angew. Chem. Int. Ed. Engl. 33:2061, 1994; and Gallop et al., J. Med. Chem. 37:1233, 1994.

Libraries of compounds may be presented in solution (e.g., Houghten, Biotechniques 13:412-421, 1992), or on beads (Lam, Nature 354:82-84, 1991), chips (Fodor, Nature 364:555-556, 1993), bacteria (Ladner, U.S. Pat. No. 5,223,409), spores (Ladner U.S. Pat. No. 5,223,409), plasmids (Cull et al., Proc Natl Acad Sci USA 89:1865-1869, 1992) or on phage (Scott and Smith, Science 249:386-390, 1990; Devlin, Science 249:404-406, 1990; Cwirla et al. Proc. Natl. Acad. Sci. 87:6378-6382, 1990; Felici, J. Mol. Biol. 222:301-310, 1991; Ladner supra.).

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their anti-neoplastic activity should be employed whenever possible.

Those skilled in the field of drug discovery and development will understand that the precise source of a compound or test extract is not critical to the screening procedure(s) of the invention. Accordingly, virtually any number of chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds.

When a crude extract is found to alter the biological activity of an CRYSTALLIN polypeptide, variant, or fragment thereof, further fractionation of the positive lead extract is necessary to isolate chemical constituents responsible for the observed effect. Thus, the goal of the extraction, fractionation, and purification process is the careful characterization and identification of a chemical entity within the crude extract having anti-neoplastic activity. Methods of fractionation and purification of such heterogenous extracts are known in the art. If desired, compounds shown to be useful agents for the treatment of vascular disease are chemically modified according to methods known in the art.

### Kits or Pharmaceutical Systems

The present compositions may be assembled into kits or pharmaceutical systems for use in ameliorating vascular disease. Kits or pharmaceutical systems according to this aspect of the invention comprise a carrier means, such as a box, carton, tube or the like, having in close confinement therein one or more container means, such as vials, tubes, ampules, bottles and the like. The kits or pharmaceutical systems of the invention may also comprise associated instructions for using the agents of the invention.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

### EXAMPLES

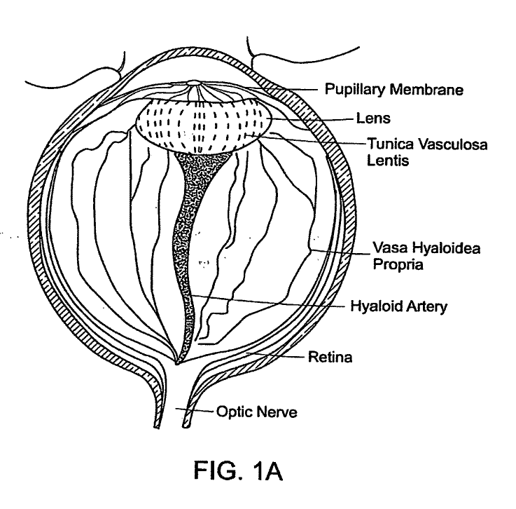

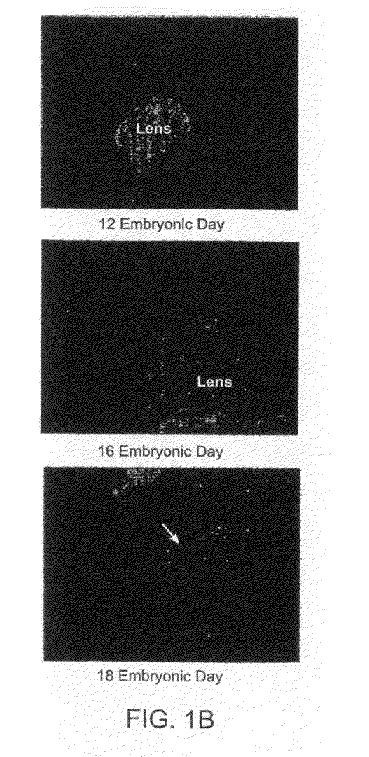

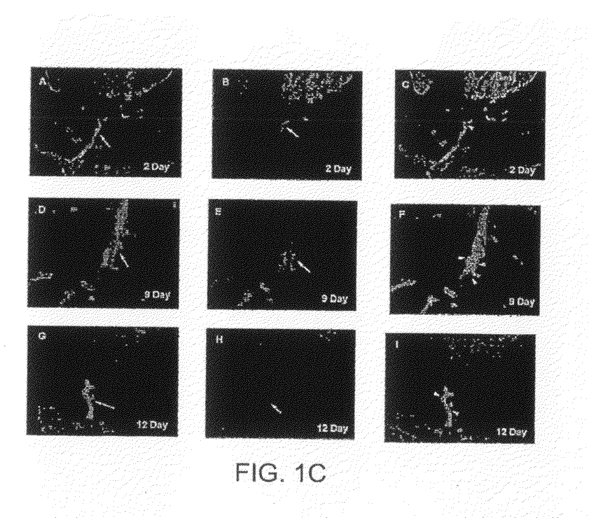

During development of the mammalian eye, nourishment of the immature lens, inner retina and vitreous is ensured by the hyaloid vascular system, including the pupillary membrane, tunica vasculosa lentis, vasa hyaloidea propria and the hyaloid artery as shown FIG. 1A. The hyaloid vessels regress by a programmed cell death process that occurs during ocular development. This occurs prior to gestation in humans and occurs within the first few weeks post-gestation in rodents. In mice, the hyaloid vessels undergo involution, starting around post-natal day 5, coincident with the onset of retinal vessel development in the inner retina. As the retina matures, the inner layers of the retina become vascularied. The mature lens and vitreous remain avascular resulting in an optically clear visual axis.

### Example 1

### β/γ-Crystallins are Expressed During Development of the Mouse Retina

Histological sections of the embryonic eyes from C57BL6 mice (12, 16 and 18 embryonic days), as well as early post natal development (2, 9 and 12 post natal days) expressed β-crystallins at different time points during development (FIGS. 1B and 1C). The expression of β-crystallins was not observed in the retina at 12 or 16 embryonic days, but by embryonic day 18 the expression in the retina (arrow) as well as in the hyaloid vasculature (FIG. 1B, asterisk) was evident (A similar expression pattern is also seen using antibodies to γ-crystallins).

### Example 2

### β/γ-Crystallin Expression is Upregulated in the Nuc1 Spontaneous Mutant Rat and the Human Condition, Persistent Fetal Vasculature (PFV)

A spontaneous mutation in the Sprague-Dawley rat with a novel eye phenotype has been reported. This mutation, Nuc1, behaves as a single semi-dominant locus with an intermediate phenotype in heterozygotes. Homozygous Nuc1 rats are fully viable and have microphthalmia, retinal abnormalities and disruption of lens structure shortly before birth.

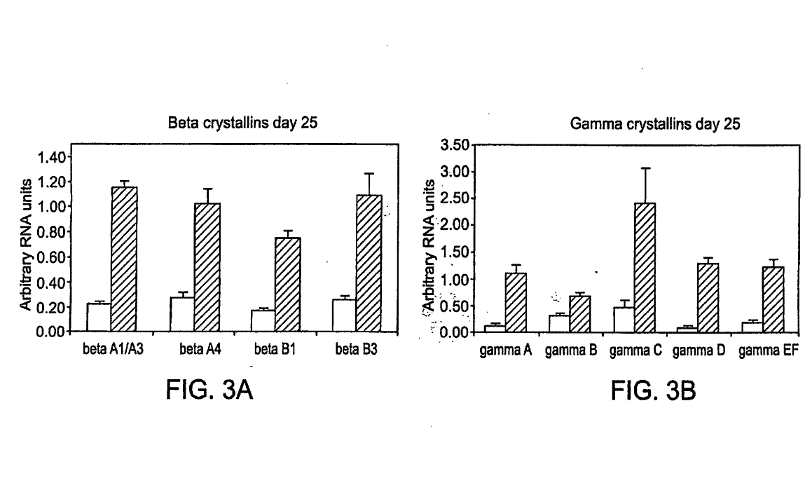

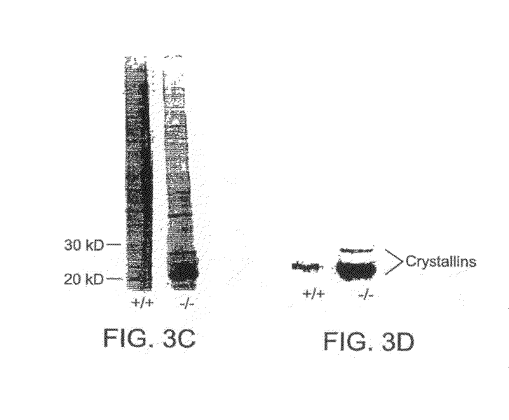

A microarray analysis was used to compare gene expression in the retinae of Nuc1 homozygous rats with wild-type littermates. Increased expression of a number of β- and γ-crystallin genes in Nuc1 was observed. The array analysis indicated more than two-fold increase in the expression of β- and γ-crystallins in Nuc1 homozygous rat retina. Real-time RT-PCR on 25-day old retinas of Nuc1 homozygous (inclusive of the hyaloid artery) and wild-type Sprague Dawley rats confirmed this upregulation. mRNA levels of β A1/A3, β A4, β B1, β B3, γ A, γ B, γ C, γ D and γ EF crystallins were significantly increased in Nuc1 retina compared to the wild-type (FIG. 3A-3D)

### Example 3

### Persistent Hyaloid Vascular System in Nuc1 Homozygous Rats

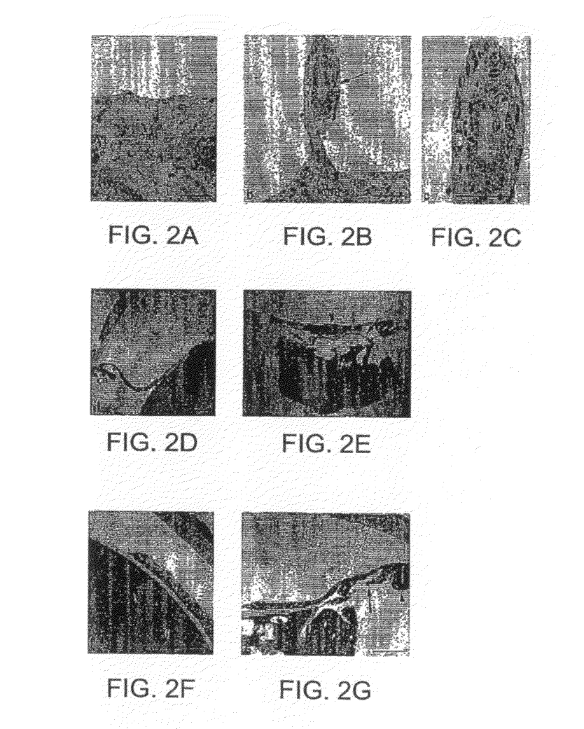

In wild-type rats, the hyaloid artery, which is a constituent of the transient vasculature that nourishes the immature lens, retina, and vitreous, regressed by day 35 (FIG. 2A). In contrast, in Nuc1 homozygous rats, the hyaloid artery persisted at the surface of the optic nerve head projecting into the vitreous until adulthood (FIGS. 2B and 2C). The pupillary membrane (PM), a temporary capillary network on the anterior surface of the lens, also known as the anterior tunica vasculosa lentis, normally regresses during the second week after birth. As shown in FIG. 2D, in the normal eye, the pupillary membrane was absent by post-natal day 25. The pupillary membrane persisted in Nuc1 at the same post-natal stage (FIG. 2E, arrows). Nuc1 rats also displayed iris hyperplasia (FIG. 2E, arrowhead) and disrupted lens structure (FIG. 2E). By four months of age, the iris and ciliary body were dragged towards the center of the posterior chamber (FIG. 2G, arrow), inducing dragging and folding of the peripheral retina (FIG. 2G arrowhead).

### Example 4

### β and γ Crystallin Expression in the Retina and Hyaloid Vessels

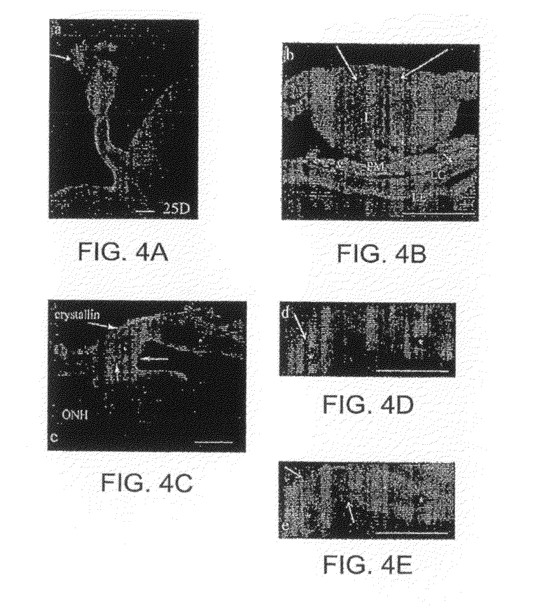

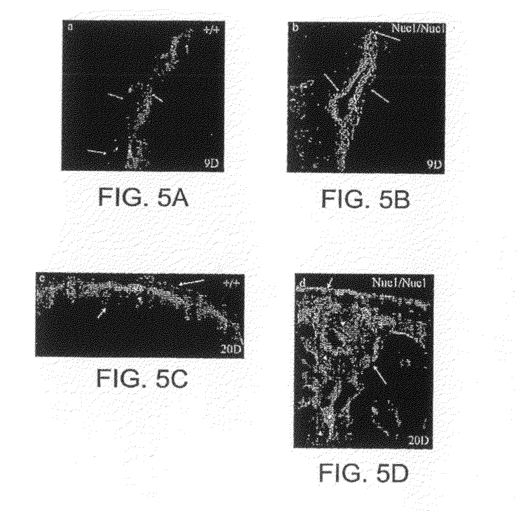

Incorporation of ³⁵S-amino acids by organ cultured 20-day retina also indicated a marked increase in crystallin synthesis in Nuc1 homozygotes compared to wild-type (FIG. 2C). Crystallin protein expression was confirmed by Western blotting (FIG. 2D). The retained hyaloid artery and vasa hyaloidea propria in Nuc1 assumed a configuration typical of the struts of an umbrella or the guide ropes of a parachute (FIG. 4A). Distinct staining patterns with the crystallin antibodies were obtained for the retained hyaloid artery (FIG. 4A,C, arrow) and pupillary membrane (FIG. 4B) in Nuc1. When the γ-crystallin staining pattern (similar data were obtained with β-crystallin) in the retained hyaloid artery (FIG. 4C, arrow) was compared to the section stained with isolectin-B4 (FIG. 4D), it clearly indicated that crystallin was localized around the blood vessels [see merged picture with γ-crystallins and Isolectin-B4 (FIG. 4E). Isolectin B4 has been shown to label blood vessels in the rat (Ashwell V is Neurosci 2:437-448, 1989). The transient hyaloid artery at post-natal day 9 in normal (FIG. 4A) and Nuc1 homozygous rats (FIG. 4B) showed distinct crystallin expression (FIG. 5A, B, arrows) surrounding the blood vessels (FIG. 5A,B, asterisk). In rats, the hyaloid artery starts involuting around post-natal day 7 and regresses completely by 3 weeks of post-natal development. While the hyaloid vessels undergo involution, the retinal vessels continue growing and reach the adult form by the time the hyaloid vasculature has completely regressed. The vascularization of the retina is restricted to the inner part of the retina. At post-natal day 20, crystallin expression is also localized surrounding the retinal vessels in both normal (FIG. 5C) and Nuc1 homozygous rats (FIG. 5D).

### Example 5

### VEGF and γ- and γ-Crystallin Expression in GFAP-Immunopositive Astrocytes of the Retained Vasculature in Nuc1 Homozygote Rats

The cellular identity of crystallin-immunopositive cells was analyzed in the retained hyaloid vasculature of the 5-week-old Nuc1 homozygote. Previous studies have shown that astrocytes migrate ahead of the vessels and are present on the external surface of the blood vessels (Stone et al., J Neurosci 15:4738-4747, 1995). Therefore, antibodies to GFAP were used to localize the astrocytes in the retained hyaloid artery and retinal vessels of Nuc1 homozygotes. VEGF has also been shown previously to be expressed by astrocytes (Wechsler-Reya et al., Curr Biol 7:R433-436, 1997) and has an important function in vascular remodeling.

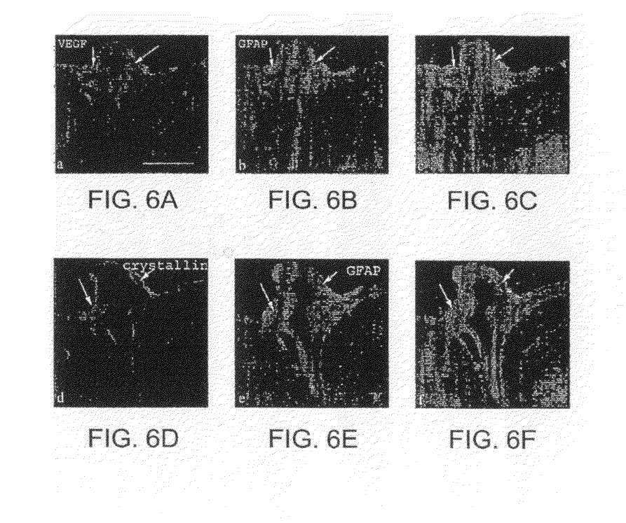

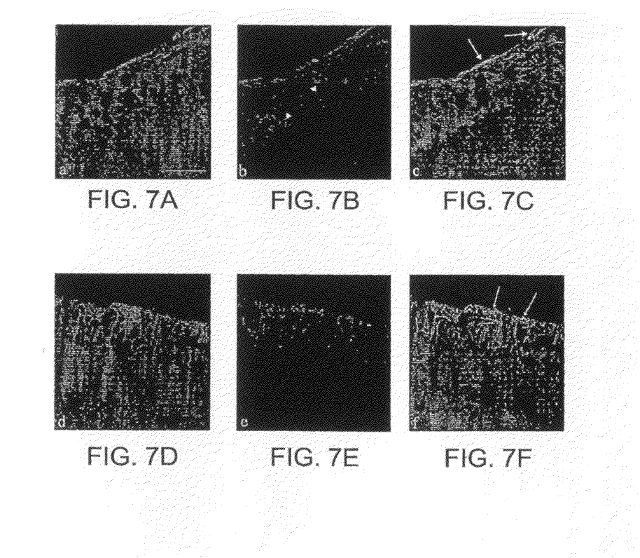

VEGF immunopositive reactivity was present in the retained hyaloid tissue of Nuc1 (FIG. 6A), co-localizing with GFAP (FIG. 6B, C). Co-localization of GFAP and γ-crystallin reactivity (FIG. 6D,F) clearly demonstrated that astrocytes in the retained hyaloid of Nuc1 also expressed crystallins. In the Nuc1 homozygous retina, confocal microscopy with crystallin specific antibodies also showed expression at the internal limiting membrane (FIG. 7B) and in ganglion cells (FIG. 7B, arrowheads). GFAP immunopositive staining was also present in the internal limiting membrane of the Nuc1 retina but not in the ganglion cells (FIG. 7A). Co-localization of crystallins and GFAP (FIG. 7C) showed that GFAP⁺ cells associated with vessels at the inner limiting membrane also expressed crystallins. VEGF expression was localized prinarily at the internal limiting membrane (FIG. 7E). A similar pattern of staining was observed with antibodies to GFAP (FIG. 7D) and co-localization indicated that GFAP⁺ astrocytes do express VEGF (FIG. 7F, arrows).

### Example 6

### VEGF and β- and γ-Crystallin Expression in Human PFV

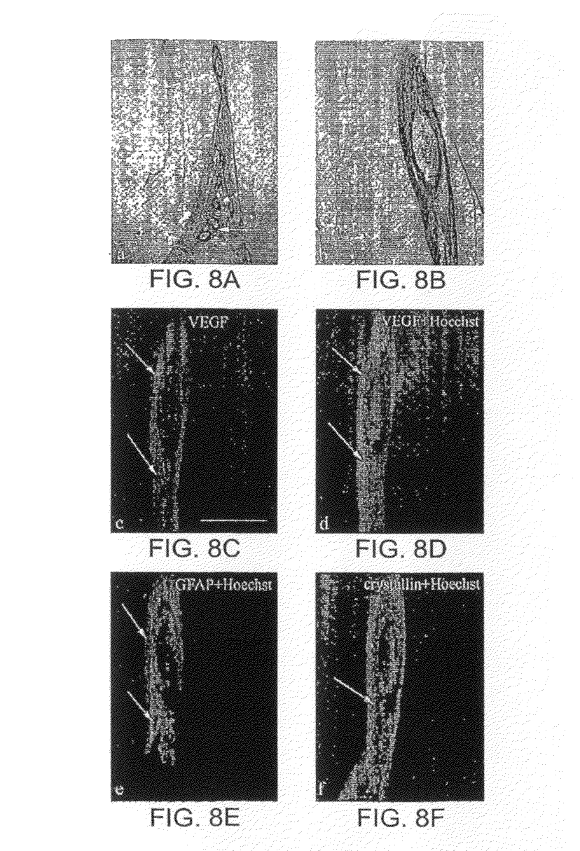

Failure of regression of the hyaloid vasculature in the human eye leads to persistent fetal vasculature (PFV) disease. The ocular phenotypes of Nuc1 and human PFV are similar. To determine if γ- and γ-crystallins are also expressed in the GFAP⁺ astrocytes of the retained hyaloid tissue of PFV patients, a similar approach was used as described above for Nuc1. VEGF, GFAP, and crystallins were immunolocalized from human PFV patients, on serial paraffin sections, all of which showed a positive staining pattern comparable to the Nuc1 rat data. FIGS. 8A-8F show representative PFV tissue section (one of five patient eyes examined) from a female who died six days after birth with a diagnosis of trisomy 13 (Patau's syndrome) with microphthalmia and multiple congenital malformations, including PFV. There was a persistent pupillary membrane, and the lens showed cataractous changes with posterior distortion. Firmly attached to the capsule was a mesenchymal fibrovascular tissue, including the well preserved hyaloid system, as shown in part in FIG. 8A and at higher magnification in FIG. 8B. Within that tissue, many well-differentiated rosettes were present (FIG. 8A, short arrows). Immunohistochemical study showed that cells surrounding the hyaloid tissue were positive with VEGF (FIGS. 8C and 8D), GFAP (FIG. 8E), and with crystallin antibodies (FIG. 8F), clearly indicating an expression pattern of VEGF and α, γ crystallins by GFAP⁺ astrocytes similar to that seen in our Nuc1 model.

### Example 7

### Induction of VEGF and β- and γ-Crystallins in Cultured Human Astrocytes Exposed to 3-Nitropropionic Acid

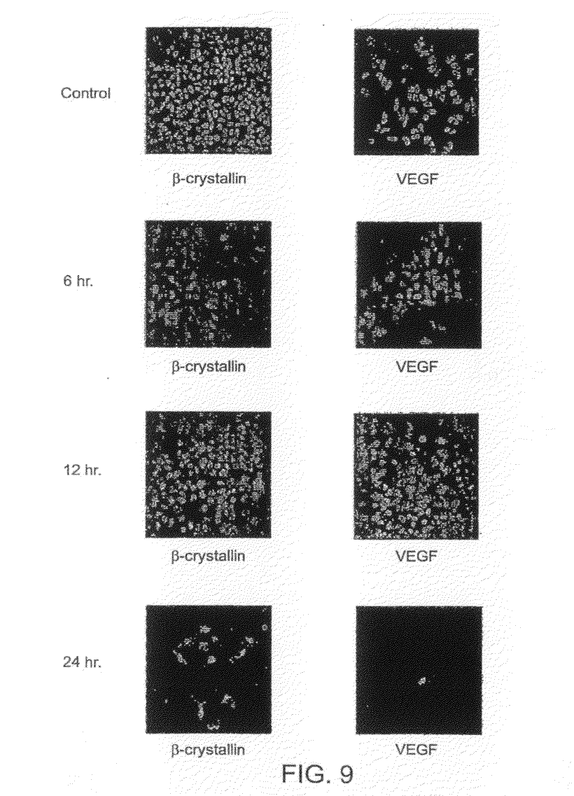

It has been shown that astrocytes are sensitive to hypoxia, which induces them to release VEGF (Chow et al., 2001; Sandercoe et al., 2003). To determine if astrocytes also express β- and γ-crystallins in response to hypoxia, 3-nitropropionic acid, an irreversible inhibitor of mitochondrial succinate dehydrogenase activity was used (Cavaliere et al., Neurochem Int 38:189-197, 2001). It can be considered a model substance to study hypoxic neuronal damage (Riepe et al., Neurosci Lett 211:9-12, 1996). As shown in FIG. 9, when fetal human astrocytes in culture were exposed to 3-NP, there was induction not only of VEGF within 6 hours, as expected, but also β- and γ-crystallins. Strong cytoplasmic staining was demonstrated with both VEGF and Bcrystallin antibodies (γ-crystallin also showed a similar staining pattern); nuclei were counterstained with DAPI. Control cells showed little or no VEGF or β, γ-crystallin staining.

### Example 8

### Abnormal Development of Retinal Cell Layers in Nuc1 Homozygous Rats

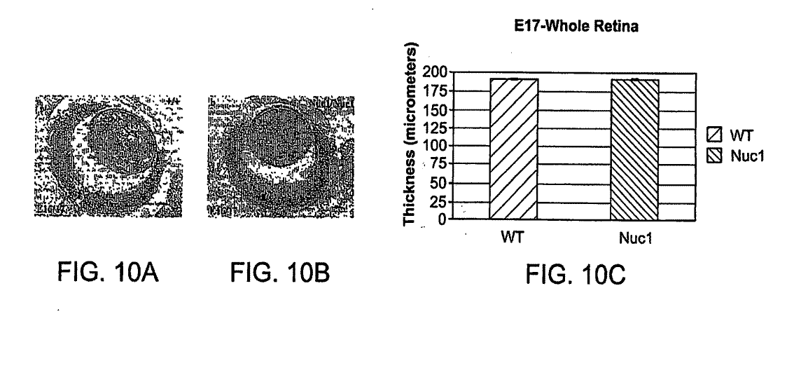

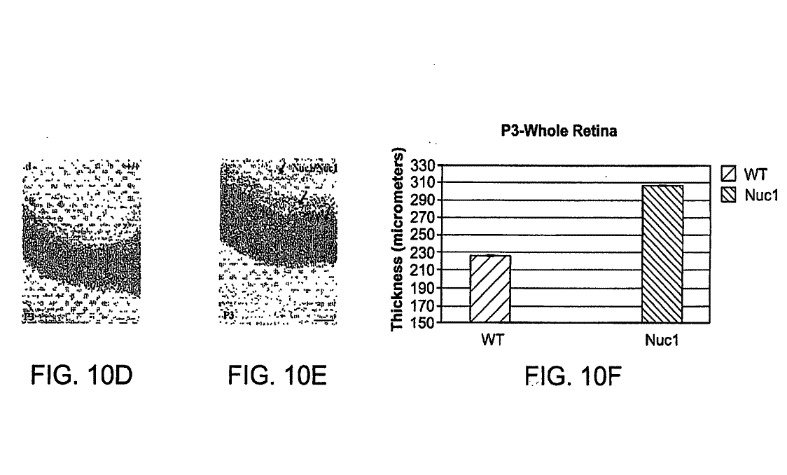

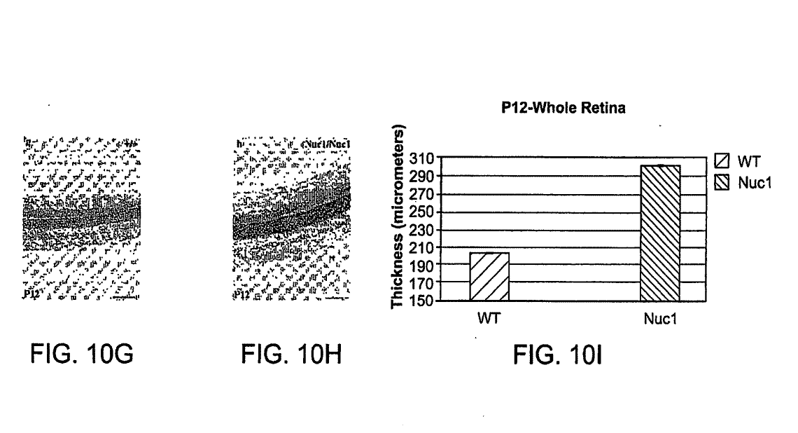

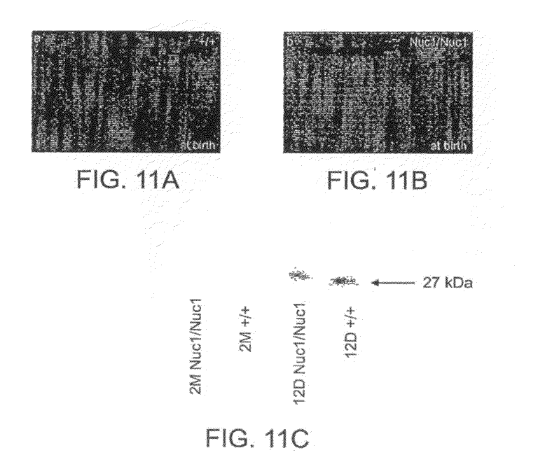

As described above, at P21 the retina of the Nuc1 homozygous rat is markedly thicker than normal. This increase resulted from increased thickness of each retinal layer rather than a particular layer, and TUNEL positive cells were decreased in the Nuc1 retina at this time. The Nuc1 retina was subsequently characterized at earlier stages of development. At E16/17, when the retina primarily consists of the inner and the outer neuroblastic layers, there was no difference in the thickness or organization of the wild-type and Nuc1 homozygote retina (FIGS. 10A and 10B). Morphometric analysis, comparing regionally matched and identically oriented sections of seven different retinas from wild-type and Nuc1 homozygotes, confirmed this finding (FIG. 10C). These studies suggested that the Nuc1 mutation did not affect the proliferation of the multipotent progenitor cells. PCNA staining was similar in the neonatal wildtype and Nuc1 homozygous retina (FIGS. 11A and 11B). Further, the expression of the cyclin-dependent kinase inhibitory protein, p27kip1, was similar at day 12 and declined by 2 months in both wild-type and Nuc1 homozygous retinae (FIG. 11C). By P3, the Nuc1 homozygote retina was thicker than normal (FIG. 10D-10F). Apoptosis is known to play an important role in the remodeling of the retina; in the mouse retina four different apoptotic waves have been identified (Pequignot et al., Dev Dyn 228:231-238, 2003). By P12, following the first apoptotic wave, the Nuc1 homozygote retina is much thicker than normal (FIGS. 10G and H). This increase in the thickness of the Nuc1 homozygote retina was significant, as shown by morphometric analysis (FIG. 101). It is evident from our studies to date that the Nuc1 spontaneous mutation affected the programmed cell death process of the retina rather than proliferation of the multipotent progenitor cells.

### Example 9

### Delayed Maturation of Retinal Neurons in Nuc1 Homozygous Rats

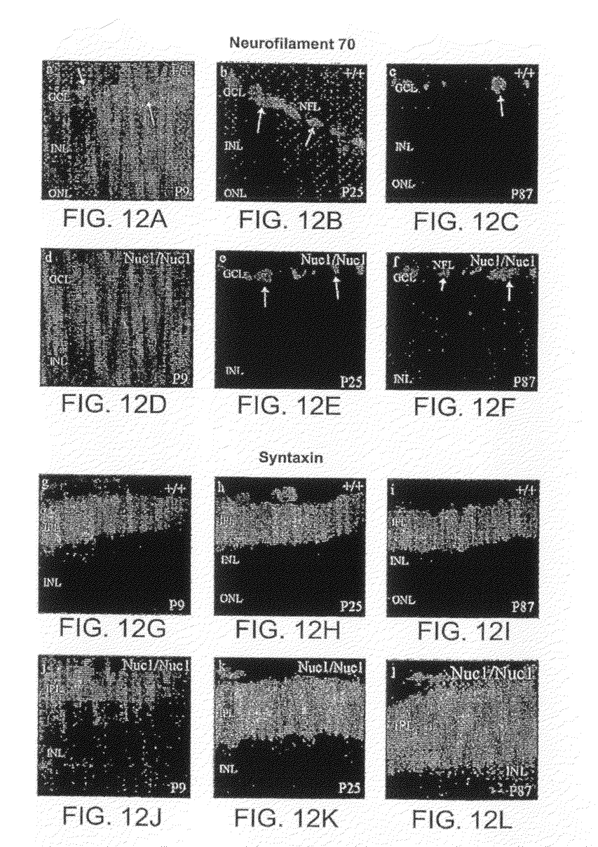

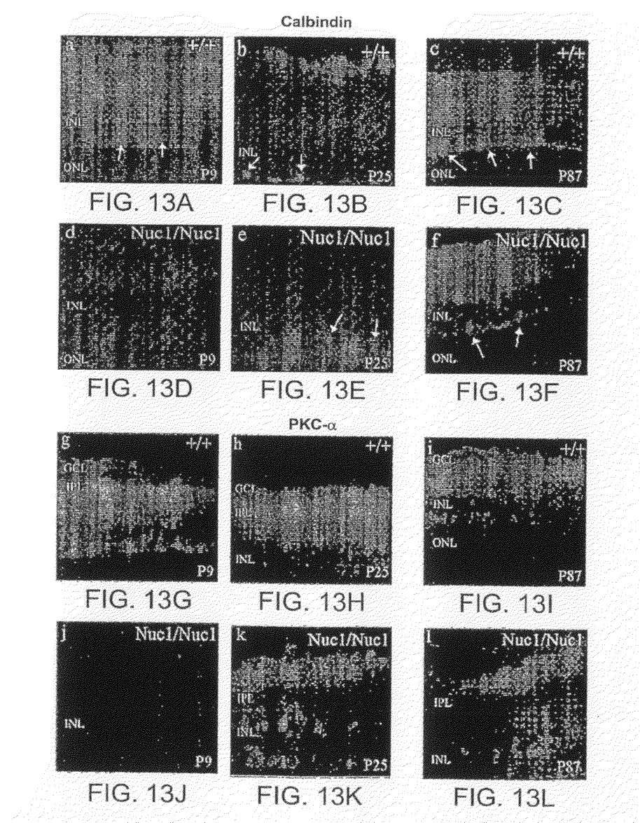

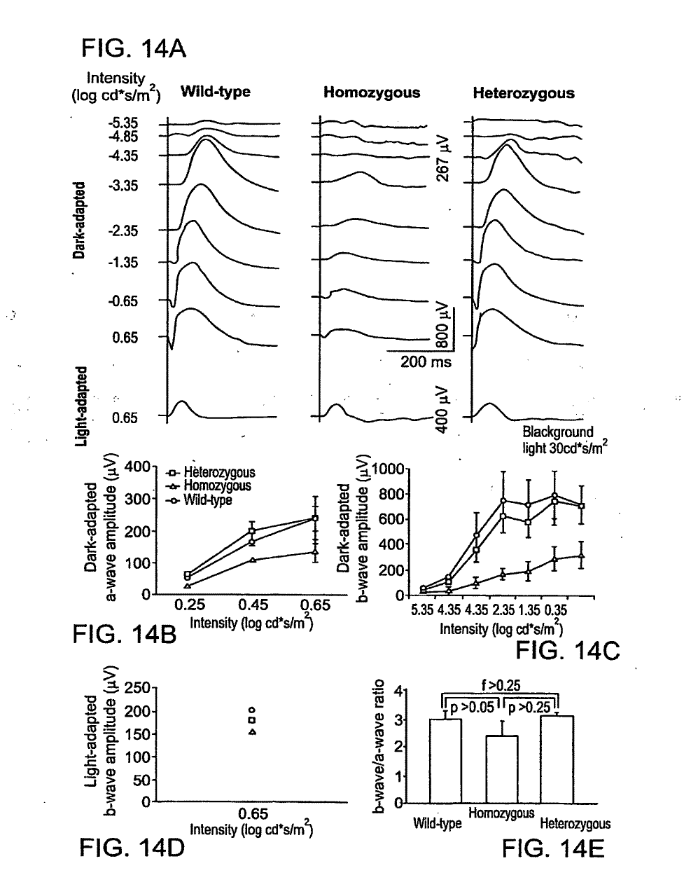

FIGS. 12 and 13 demonstrated delayed maturation of retinal neurons in Nuc1 homozygous rats. Ganglion cell differentiation was compared in wild-type and Nuc1 homozygous rats by immunohistochemistry using an antibody to the specific ganglion cell marker, neurofilament 70 (FIG. 12A-12F). Ganglion cells were clearly labeled at P9 in wild-type retina (FIG. 12A, arrows), while little, if any, reactivity was evident in Nuc1 (FIG. 12D). By P25, both wild-type and Nuc1 homozygous retina were strongly positive for the marker (FIGS. 12B and 12E, arrows). At P87 Nuc1 homozygous rats showed more neurofilament 70 expressing ganglion cells as compared with wild-type, further indicating a delay of maturation of ganglion cells in Nuc1 homozygotes (FIGS. 12C and 12F, arrows). Immunolabeling with anti-syntaxin (FIGS. 12G-12L) showed a similar delay in the differentiation of amacrine cells. Amacrine cell processes found in the IPL express syntaxin and are clearly labeled at P9 in the wildtype (FIG. 12G), but strong staining is not detected in Nuc1 homozygous rats until P25 (FIG. 12K). At P25 and P87, the Nuc1 homozygous retina appeared to have a denser population of labeled amacrine cells than the wildtype, as well as a markedly thicker IPL (FIGS. 12I and 12L). An antibody to calbindin was used to compare horizontal cell differentiation in wildtype and Nuc1 homozygotes (FIG. 13A-13F). Horizontal cells are clearly labeled with anti-calbindin at P9 in the wildtype retina (FIG. 13A, arrows), but not until P25 in Nuc1 homozygotes (FIG. 13E, arrows). Horizontal cell labeling decreases in both P87 wild-type and Nuc1 homozygote retinas (FIGS. 13C and F, arrows). Bipolar cells were visualized clearly with PKC-α at P9 in wild-type (FIG. 13G), but not until P25 for Nuc1 homozygotes (FIG. 4K). Overall, there is a decreased staining of bipolar cells in Nuc1 homozygotes as compared with wild-type (FIGS. 13I and 13L).

### Example 10

### ERG of Nuc1 Homozygous Rats

Typical wave-form morphology shows diminished a-wave and b-wave amplitudes in homozygous but not heterozygous animals, as compared with wild-type at 10 weeks. Light-adapted wave forms are preserved in all animals (FIG. 14A). The dark-adapted a-wave amplitudes of the Nuc1 homozygous rats were consistently diminished as compared with wildtype and Nuc1 heterozygotes (FIG. 14B). Dark-adapted b-wave amplitudes were significantly diminished in Nuc1 homozygous rats as compared with wildtype and Nuc1 heterozygotes at all tested flash intensities (P_{—}0.01) (FIG. 14C). The light-adapted b-wave amplitude of Nuc1 homozygotes was not significantly different than the wild-type and Nuc1 heterozygotes (FIG. 14D). The dark adapted ERG b-wave/a-wave ratio of the Nuc1 homozygous rats was also smaller than that of the wild-type and the Nuc1 heterozygotes (FIG. 14E).

### Example 11

### Fate of Rod and Cone Photoreceptors

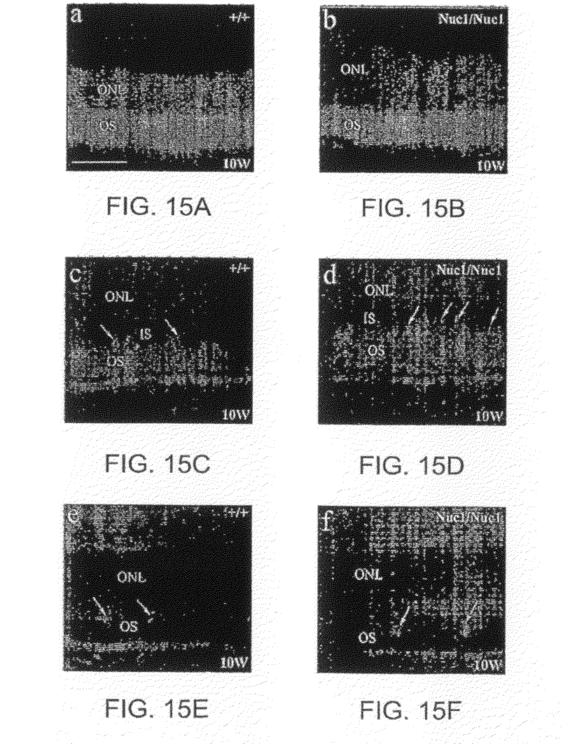

Immunohistochemistry showed abnormal rhodopsin kinase 1α staining of rod cells in 10 week old Nuc1 homozygous rats as compared with wild-type (FIGS. 15A and B). Nuc1 homozygous rats appear to have less rhodopsin kinase 1α staining of the rod outer segments compared with age-matched wild-type rats. Nuc1 homozygous retinas also showed diminished dark-adapted a- and b-waves compared with wild-type (FIGS. 15A-15F) JH455 (s cone) and JH492 (m cone) staining is present and shows relatively similar staining patterns in 10 week old wild-type and Nuc1 homozygous rats (FIG. 15C-F). These immunofluorescent data are consistent with the light-adapted b-wave ERG data that do not detect reduced cone function in the Nuc1 homozygotes (FIG. 15) during postnatal development.

### Example 12

### Retinal Thinning and Degeneration in Aging Nuc1 Homozygous Rats

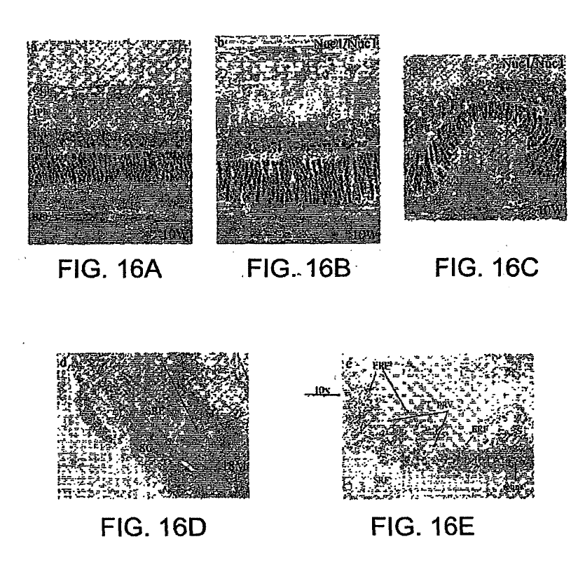

Histological analysis of wild-type and Nuc1 homozygous retinae at P75 still indicated a thickening of all retinal layers in Nuc 1 homozygotes (FIGS. 16A and B) with areas of focal detachment (FIG. 16C). At later ages the retina in Nuc1 homozygotes becomes thinner and undergoes progressive detachment. The detachment was associated with abnormalities in the pre-retinal vasculature and epiretinal fibrosis similar to traction detachments seen in advanced proliferative diabetic retinopathy (FIGS. 16D and E). While detachment may result in secondary degeneration of the retina, ERG indicated that functional retinal degeneration preceded mechanical detachment of the retina in these animals. Infiltration of macrophage like cells in the sub-retinal space of the Nuc1 homozygous retina at the time of retinal detachment was previously observed (Hose et al., Immunol Lett 96:299-302, 2005).

### Example 13

### Activation of Muller Glia and Upregulation of GFAP in Nuc1 Homozygous Rats

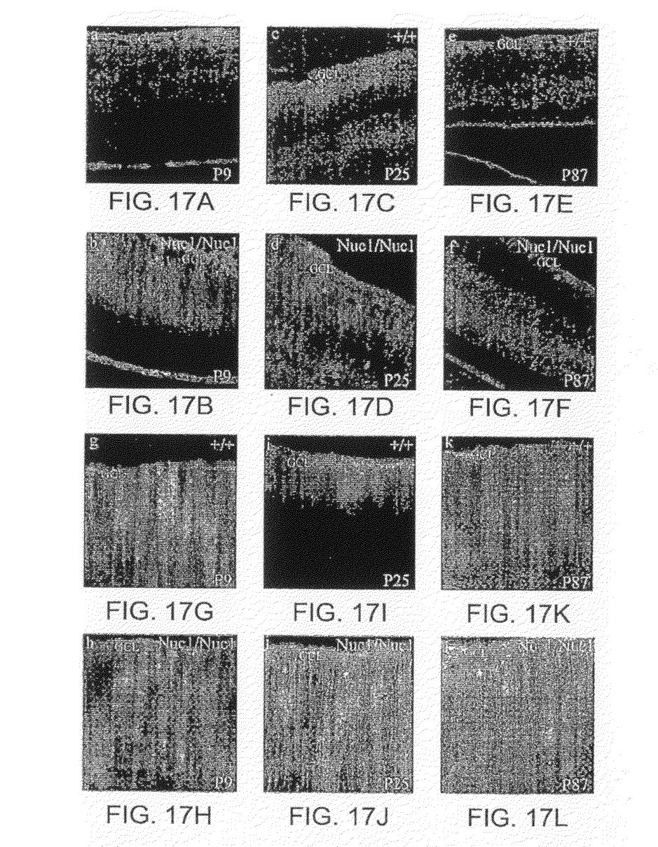

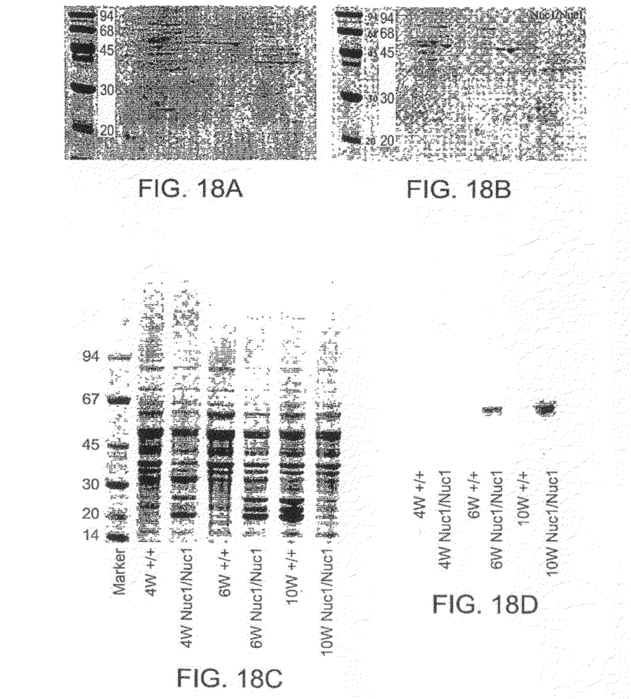

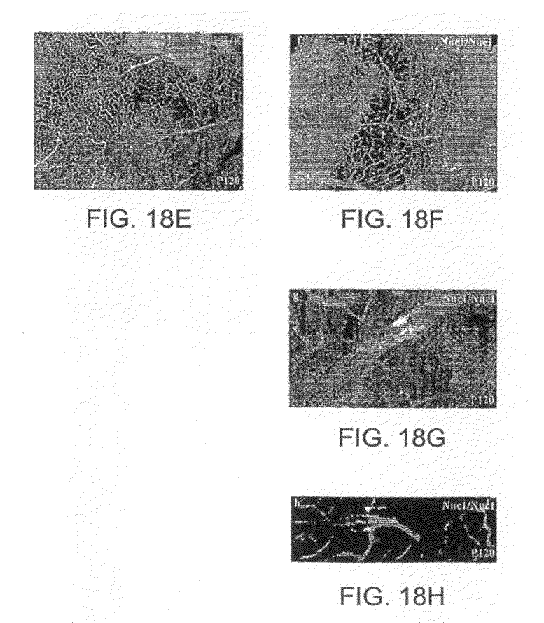

Using CRALBP as a marker, an increase in the staining of Muller cells in the Nuc1 homozygous retina was observed (FIGS. 17A-F). The staining was much more intense at all three time points tested in Nuc1 homozygotes (FIGS. 17B, D and F, asterisks) compared with wild-type retina. Muller glia are also known to express GFAP under certain conditions, particularly in response to stress or injury (Levine et al., Dev Biol 219:299-314, 2000). Labeling with GFAP at P9, P25 and P87 showed an increase in the Muller cell staining pattern in Nuc1 homozygotes, with more intense staining of Muller cell processes, which can be seen to extend into the retina from the inner limiting membrane at P25 and P87 (FIGS. 17J and L, asterisks). Two-dimensional gel electrophoresis was used to compare the expression of retinal proteins in 2 month old wild-type and Nuc1 homozygote rats. These results are shown in FIG. 18A (wildtype) and FIG. 18B (Nuc1 homozygote). In general, the patterns of the two gels are very similar but with obvious changes in the expression profile. A few of the spots were identified as indicated by gray numbers in FIG. 18B by mass spectrometry. Using MALDITOF mass spectrometry, one of the spots (#1 in FIG. 18B) that showed an increase in intensity in the Nuc1 homozygotes was identified as GFAP.

A purified mouse anti-GFAP cocktail antibody was used to confirm upregulation of GFAP in Nuc1 homozygote rats at P45 (6 weeks) and 75 (10 weeks) by Western Blotting (FIG. 18C-D).

### Example 14

### Developmental Abnormalities in Retinal Blood Vessels of Nuc1 Homozygotes

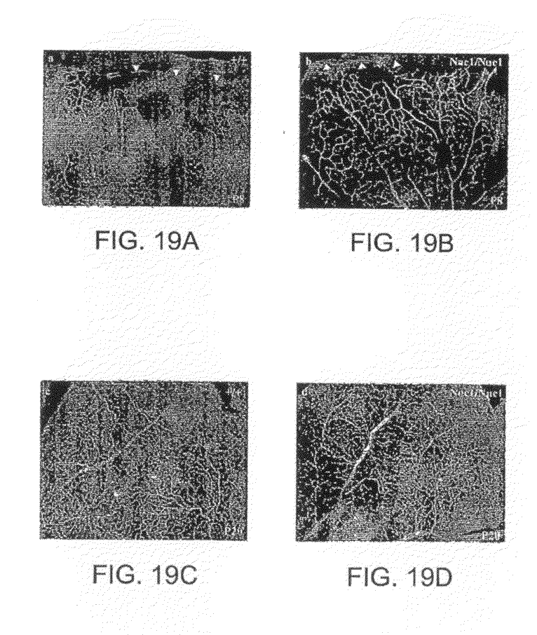

In this study, fluorescein isothiocyanate (FITC)-dextran perfusion suggested that fully vascularized retina reaches the ora serrata in P8 Nuc1 homozygous rats in advance of wild-type rats. Once fully vascularized, the early vascular patterning in the retina was similar in P20 Nuc1 homozygous and wildtype rats, apart from a few vessels with increased caliber in Nuc1 homozygotes (FIGS. 19A-19D). As the Nuc1 homozygous rat matured, the vascular architecture and patterning become increasingly abnormal (FIGS. 19E and 19F). Microaneurysm formation and vascular dropout in the retinal blood vessels of Nuc1 homozygous rats are observed at P120 (FIG. 19F, arrows), but not in wild-type (FIG. 19E). Abnormal hyperfluorescence inside vessels was also observed (FIG. 19G, asterisks). Blockage of blood flow inside some vessels (FIG. 19H) was associated with vascular dropout at P120 in Nuc1 homozygotes.

In summary, these studies showed that the development of the retinal neurons, Muller glia, and the vasculature is abnormal the Nuc1 homozygous rat. Functional deficits, particularly in the rod photoreceptors, were evident by ERG.

### Example 15

### β/γ-Crystallins Regulate the Function of Cells Involved in Vascular Remodeling in a Cell-Culture System

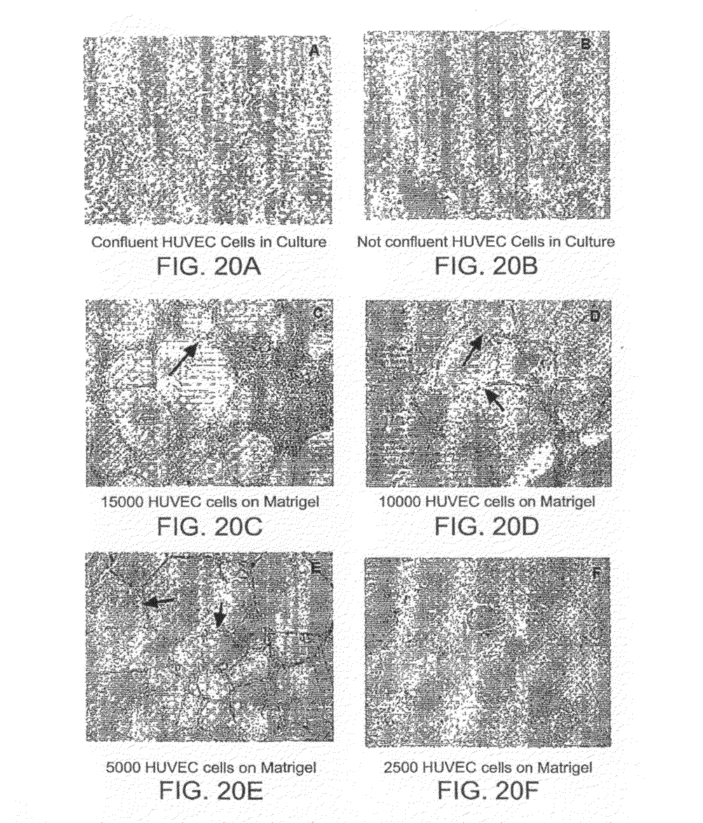

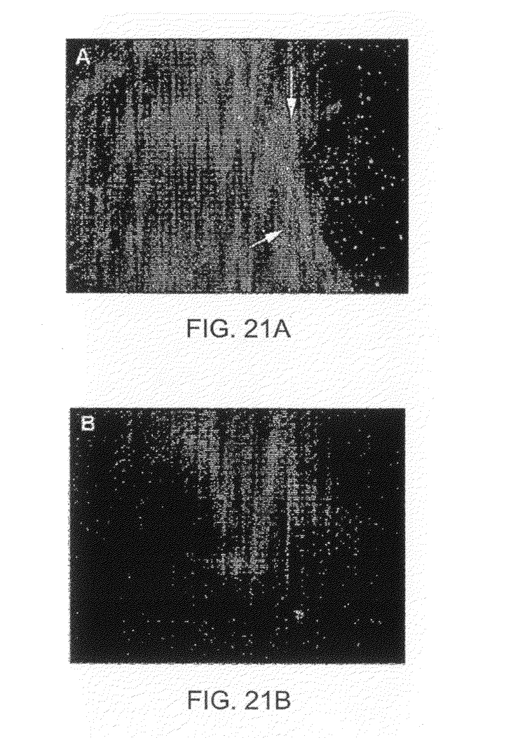

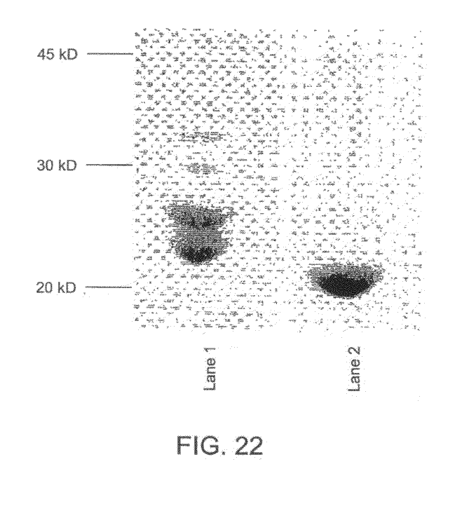

In order to better understand the functional role of β/γ-crystallins in vascular remodeling, HUVEC (Human umbilical vein endothelial cells) were established in culture. HUVEC differentiation into tube-like structures in three dimensional Matrigel cultures has been commonly used as a model system to studying angiogenesis (Edelman et al. Exp. Eye Res. 80(2): 249-58, 2005; Xin et al. J. Biol. Chem. 274(13): 9116-21, 1999). In functional studies with β, γ-crystallins and VEGF the seeding density used is shown in FIG. 20. After plating HUVEC onto Matrigel, the cells differentiated to form tubes and a capillary-like network at the minimum seeding density of 5×10³ cells/well (FIGS. 20A-20F). These differentiated cells form tubes and express crystallins (FIGS. 21A and 21B).

The effect of β/γ crystallins and VEGF on HUVEC tube formation and regression was examined. VEGF protein used in this study was purchased from R&D Systems (Minneapolis, Minn.) and the polyclonal antibody was from Santa Cruz Biotechnology (Santa Cruz, Calif.). To prepare the crystallin proteins, lenses dissected from bovine eyes were homogenized in 7 volumes of 0.05M Tris buffer, pH 7.4 containing 0.1M KCl, 10 mM 2-mercaptoethanol and 0.02% NaN₃. After centrifigation at 27,000×g for 15 minutes at 4° C., aliquots of the supernatant were loaded onto a Superose 12 HR column (Amersham Biosciences) pre-equilibrated with the same buffer. The column was run at 0.5 ml per minute on an Agilent Model 1100 HPLC. Fractions were collected and peaks analyzed by SDS-PAGE. Fractions from peaks of interest were pooled, dialyzed against water, lyophilized and then re-dissolved in the chromatography buffer. β-crystallin peaks were re-run on the same column and γ-crystallin peaks on a Superdex 75 column (Amersham Biosciences). Fractions from the rechromatographed peaks were assayed by SDS-PAGE and those fractions free of contaminating crystallins were pooled, dialyzed against water, and lyophilized. The lyophilized preparation of isolated β_{H}, β_{L} and γ-crystallin were kept dry at −20° C. until used. FIG. 22 shows gel patterns of purified crystallin proteins used in our studies. Antibodies against purified crystallin proteins were raised in rabbits. The polyclonal antibodies have been used earlier for Western blotting and immunohistochemistry (Thang et al. Dev Dyn. 234(1): 36-47, 2005) have been found to be very specific.

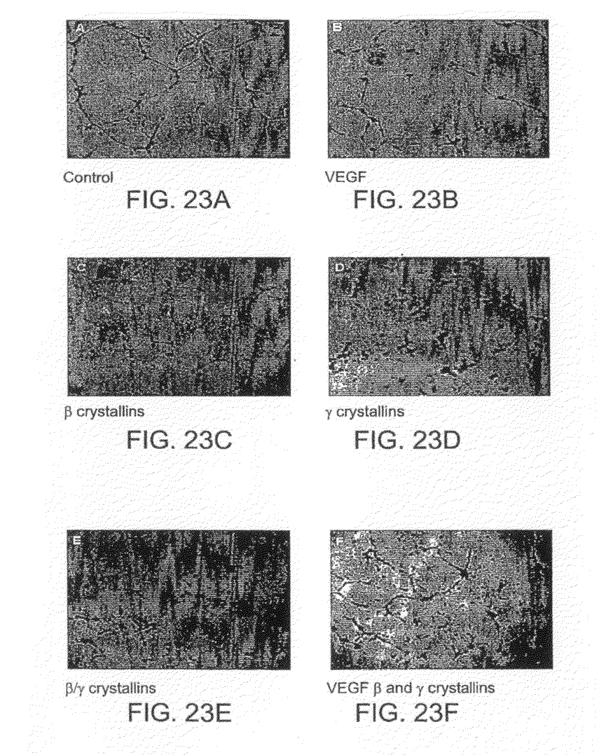

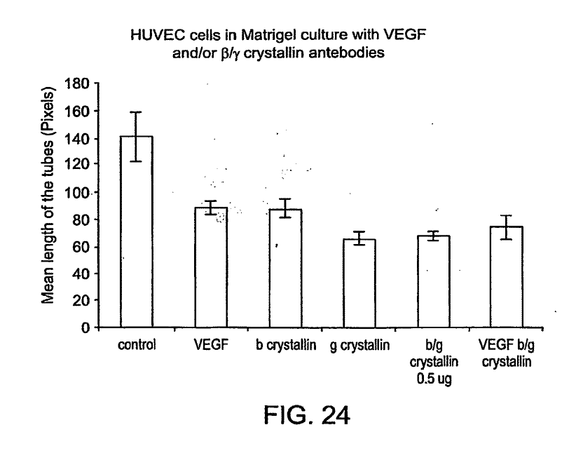

The three-dimensional (3D) tube formation assay of HUVEC was used to demonstrate the effect of crystallins on tube formation. The HUVEC were exposed to antibodies against β-crystallin or γ-crystallin or both P and γ-crystallins at the time of plating. Antibodies against VEGF were used as the positive control. Inactivated normal rabbit serum served as the negative control. The cells were exposed to 0.5 μl to 5 μl of crystallin antisera. Maximum effect with negligible cell death as determined by trypan-blue exclusion was optimal with 1 μl and hence 1 μl of antibody was used in the present experiment. After plating HUVEC onto Matrigel, the cells attached to the matrix and morphologically differentiated to a capillary-like network (tubes) in about 24 hours. When compared to controls, β,β-crystallin and VEGF antibody treated HUVEC exhibited reduced tube formation. Semi-quantitative analysis performed by measuring the length of all the tubes in three different fields, on seven different culture plates indicated reduced length compared to controls (FIGS. 23 and 24).

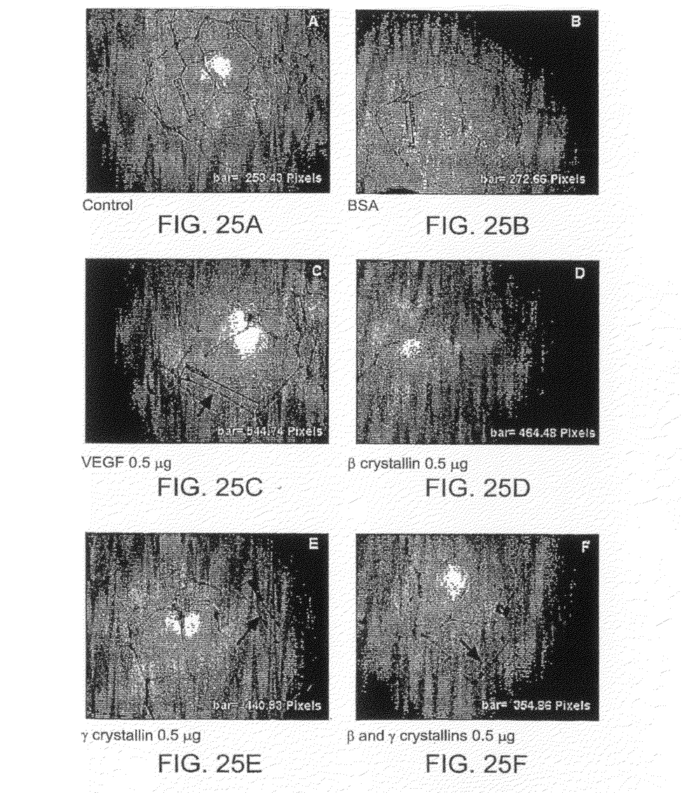

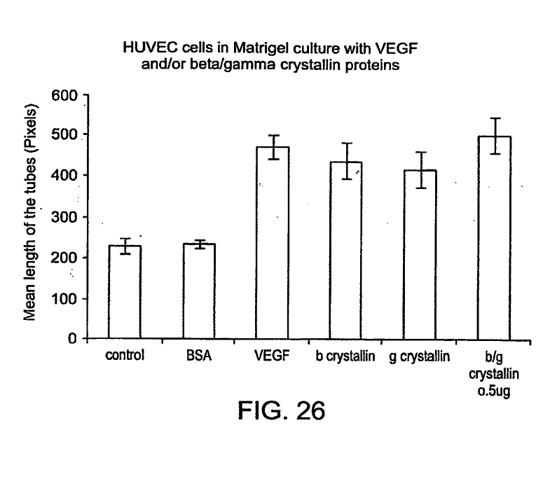

Interestingly, when HUVEC in culture were exposed to purified β/γcrystallin proteins (0.5 μg/ml), they showed much longer tube processes compared to controls as shown in FIGS. 25 and 26. The cells were exposed to 0.2 μg/ml to 2 μg/ml of crystallin proteins, with a maximum effect at 0.5 μg/ml with negligible cell death as determined by trypan-blue exclusion. 0.5 μg/ml of crystallin protein concentrations were used in the present experiment.

It has been postulated that “physiological hypoxia” is required for formation and survival of the transient embryonic vasculature of the eye. Physiological levels of hypoxia are the stimulus for normal development of the tallins in the retinal vasculature. Pathological hypoxia serves as a stimulus for neovascular disease of the retina. Astrocytes have multiple functions that include regulation of blood vessel structure and function. These results indicated that β/γ-crystallins are expressed by astrocytes that surround developing blood vessels and also by vascular endothelial cells. To determine whether hypoxia induced crystallin synthesis in astrocytes, human astrocytes were cultured exposed to 3-nitropropionic acid (3-NP), a substance shown to induce neuronal hypoxia. The astrocytes secrete VEGF and γ/γ-crystallins within 6 hours of exposure. It is known that astrocytes respond to the changing physiological levels of hypoxia during development. Temporally coincident increases in expression of VEGF and the β/γ-crysin astrocytes could be the result of common regulatory elements or pathways. Although VEGF is known to be regulated by hypoxia, this 3-NP data suggested that hypoxia regulated the β- and γ-crystallins.

### Example 16

### γ Crystallins Directly Interact with VEGF

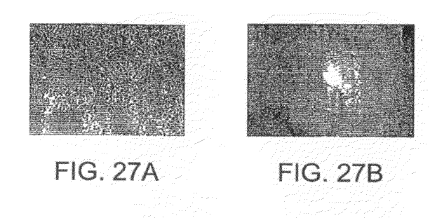

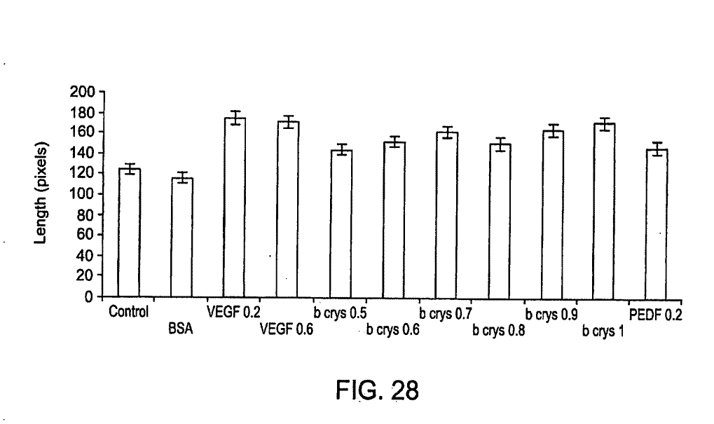

As reported above, a functionally significant effect of β/γ crystallins on vascular remodeling the effect of β/γ crystallins was established on human umbilical vein endothelial cells (HUVEC) in culture. Because the behavior of vascular endothelial cells varies according to their source and the resident tissue environment, the effect of β/γ crystallins on cultured human retinal endothelial cells (HREC) was subsequently analyzed. Exogenously applied β/γ crystallins significantly enhanced HREC vessel-like tube formation on tissue culture plates pre-coated with Matrigel (FIGS. 27A and 27B). Tube formation was also enhanced in the presence of exogenously added vascular endothelial growth factor (VEGF). FIG. 28 quantifies these effects. Most striking is that the effect of β/γ crystallins is comparable to that of VEGF at the concentrations tested. (PEDF at the relatively high dose tested is known to stimulate tube formation while at lower concentrations inhibition occurs.)

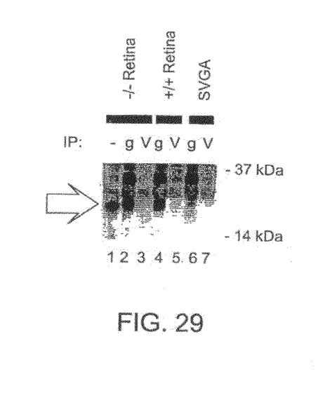

In view of evidence of a direct functional effect of β/γ crystallins in vitro on HUVEC and HREC and the observation that both VEGF and crystallin are selectively over expressed in the Nuc1 (Sprague-Dawley mutant rat) homozygote lenses as compared to normal Sprague-Dawley rats, possible relationships between VEGF and crystallins were examined. FIG. 29 shows results from co-immunoprecipitation experiments that indicated a molecular interaction between γ crystallins and VEGF. Polyclonal antiserum raised against purified γ crystallins, was able to detect a γ crystallin band immunoprecipitated from retinal lysates of 23 day-old Nuc1 homozygous rats with anti-VEGF antiserum (FIG. 27, lane 3), but no such band was detectable in anti-VEGF immunoprecipitates of the 23 day wild-type retinal lysate (lane 5), or confluent human astrocyte lysate (lane 7) where VEGF levels would be expected to be lower. SVGA cells do express higher levels of both VEGF and γ crystallins under hypoxic conditions.

Finally, several members of this protein family incorporate “RGD” or closely-related sequences into regions of their secondary structure that are likely to be freely soluble and thus accessible for protein-protein interactions. RGD sequences are established binding targets for several integrin proteins, which are important constituents of the VEGF-sequestering extracellular matrix. Members of this family have been firmly implicated in angiogenesis, among other cell functions, and thus could play a role in mediating the cellular activities of β/γ crystallins.

In view of these results, the β/γ crystallins likely play a functional role in ocular vascular remodeling.

The results reported herein were obtained using the following methods and materials.

### Specimen Preparation

Experiments were performed on post-natal or embryonic Nuc1 mutant and wild-type Sprague Dawley rats, as described by Sinha et al., Exp Eye Res 80:323-335, 2005. Post-mortem samples were fixed in 10% formalin, and 5-μm sections were cut and either stained with PAS or processed for immunofluorescence.

Nuc1 mutant and wildtype Sprague Dawley rats were selected at ages from E16/17 to P120. The Nuc1 homozygote progeny used in this study were obtained by brother-sister mating. Timed pregnancy wild-type Sprague-Dawley rats were purchased from Taconic Farms, Germantown, N.Y., USA for age matched studies.

### Real Time RT-PCR

Real time RT PCR was used to determine the expression of γ and γ-crystallins in wild type and Nuc1 homozygote retinas. Total RNA from samples was reverse transcribed using Super Script II Reverse Transcriptase (Invitrogen, La Jolla, Calif.). For Real-time PCR analysis, LightCycler FastStart DNA Master SYBR Green kit (Roche Diagnostics) and the Light Cycler from Roche Diagnostics were used. Primer sets for β- and γ-crystallin family members were taken from published sequences and are available upon request. Since β- and γ-crystallin family members share close homology, we selected non-homologous regions using the ComAlign software. Primer pairs based on the respective rat sequences of each crystallin gene from the Ensembl database were then designed using the primer 3 software. Hypoxanthine PhosphoRibosyl Transferase (HPRT) was used as an internal control. SYBR green was incorporated into the reaction mixture to facilitate measurement of product. The integrity of PCR product was verified by melting curve analysis. Real-time PCR values were determined by reference to a standard curve that was generated by Real-time PCR amplification of serially diluted cDNAs using β- and γ-crystallin and HPRT primers. Values obtained for levels of β and γ-crystallins were normalized to the levels of HPRT mRNA.

### Metabolic Labeling

To determine if new synthesis of crystallins in Nuc1 homozygotes was different from that in the wild type, 20-day-old retinas in organ culture were incubated with 200 μci of ³⁵Slabeled amino-acids (Easy Tag Protein labeling mix, Perkin Elmer Life Sciences, Oak Brook, Ill.) for 3 hours. Retinal samples were then rinsed and homogenized in 20 mM Tris (pH 7.1) containing protein inhibitors (Roche) in a loose-fitting plastic tissue grinder to disrupt cells, but not to disrupt the nuclei. The samples were centrifuged at maximum speed in an Eppendorf microfuge for 15 minutes to remove debris and cell nuclei. To each supernatant was added a few microliters of DNase (Roche) to digest any remaining DNA. After 30 minutes of incubation at room temperature, SDS sample buffer with reducing agent (Novex) was added. The samples were placed in a boiling water bath for 2 minutes and then loaded on a Nu-PAGE (4-12%) Bis-Tris gradient gel (Invitrogen). Gels were stained with Coomassie brilliant blue, dried, and autoradiographed using Kodak Biomax film.

### SDS-PAGE and Western Blot Analysis

The eyes were enucleated from 20-day-old wild-type and Nuc1 homozygous rats after euthanization. The retina from each eye was dissected and rinsed in PBS and homogenized in SDS sample preparation buffer. After the supernatant fractions were heated in a boiling water bath for 2 min, approximately 100 μg protein from each preparation was loaded on 4-12% Bis Tris Nu-PAGE gels (Invitrogen). The gels were stained with Coomassie brilliant blue. For Western blotting, proteins were transferred to Nitrocellulose membranes (Bio-Rad Laboratories, Richmond, Calif.), blocked with 10% milk diluent, and incubated with the primary antibody overnight at 4° C. HRP-conjugated secondary antibodies and 4-CN substrate (Kirkegaard and Perry Laboratories) were used for visualization. A cocktail containing β- and γ-crystallin antibodies, each at 1:800 dilution was used. The antisera were raised in rabbits using calf β and γ-crystallin protein as antigen.

Secondary antibodies used were donkey anti-mouse IgG conjugated with Cy-3 (Jackson Immunoresearch, West Grove, Pa., USA) for PCNA, neurofilament 70, syntaxin and rhodopsin kinase 1α, and donkey anti-rabbit IgG conjugated with Cy-3 (Jackson Immunoresearch) for GFAP, PKC-α, calbindin, JH455 and JH492. Both secondary antibodies were used at a 1:100 concentration for this study. The sections were then examined on a Zeiss Axioskop II. Confocal microscopy was done on a Zeiss LSM510.

### Immunofluorescence

Immunofluorescence was performed on frozen sections as described earlier (Sinha et al., Exp Eye Res 80:323-335, 2005). Briefly, the sections were incubated with phosphate-buffered saline (PBS), containing 5% normal donkey serum, for 30 minutes prior to being incubated overnight with primary antibodies at 4° C., washed in PBS (PBS), incubated for 1 hour at room temperature with secondary antibodies, washed again with PBS and mounted with Vectashield medium with 4=-6-diamidino-2phenylindole (DAPI) (Vector Laboratories, Inc, Burlingame, Calif., USA).

Primary antibodies included the following: β- and γ-crystallin (1:500), VEGF (Santa Cruz, sc-152; 1:100), GFAP (glial fibrillary acidic protein) (Dako; 1:1,000) antibodies were used for single labeling; the mouse monoclonal GFAP (Santa Cruz; 1:200) was used for double labeling; crystallin antibodies were also immunoabsorbed with respective crystallin proteins and used as an additional control in the present study-, primary monoclonal mouse antibodies antibodies against PCNA (2 μg/ml; Stressgen, Victoria, BC, Canada), neurofilament 70 (1:1000; Chemicon International, Temecula, Calif., USA), syntaxin (1:6000; Sigma, St Louis, Mo., USA), cellular retinaldehyde binding protein (CRALBP) (1:1000; Affinity Bioreagents, Golden, Colo., USA) and rhodopsin kinase 1α (1:10,000; Sigma, St. Louis, Mo., USA) were used; primary polyclonal antibodies used were glial fibrillary acidic protein (GFAP) (1:1000; DAKO Corporation, Carpentaria, Calif., USA), protein kinase chain alpha (PKCe) (1:2000; Sigma), calbindin (1:1000; Sigma) JH455 and JH492 (both 1:400; both from Dr. Jeremy Nathans, Johns Hopkins University, Baltimore, Md., USA). Secondary antibodies used included the following: donkey anti-rabbit secondary antibodies conjugated to either Cy-2 or Cy-3 (Jackson ImmunoRes, West Grove, Pa., 1:200); for double labeling with biotinylated isolectin B4 (Sigma, St. Louis, Mo.) and crystallin, streptavidin-Cy2 and Cy3 conjugated secondary antibodies (Jackson ImmunoRes, 1:200) were used. For double labeling with other primary antibodies (two primary antibodies from two different species), Cy2 or Cy3 conjugated secondary antibodies were used. The sections were finally counterstained with Hoechst and mounted with DAKO fluorescent mounting medium.

### Retinal Blood Vessel Visualization

For visualization of blood vessels, rats were anesthetized and perfused with PBS containing 50 mg/ml of fluorescein-labeled dextran (average molecular weight 500,000; Sigma, St. Louis, Mo.) as previously described (To be et al., Invest Opthalmol V is Sci 39:180-188, 1998). The eyes were removed, immersed in OCT compound without fixation, and sectioned. The 7-μm sections were then immunolabeled with either β- and γ-crystallin antibodies (1:1,000 dilution). Fluorescent digital images were taken with a Zeiss microscope (Axioskop II). Confocal microscopy was done on Zeiss LSM 510.

### Histology

For hematoxylin and eosin (H & E) staining, heads of embryos or enucleated eyes from postnatal rats obtained after killing, were fixed initially in 2.5% glutaraldehyde followed by 10% buffered formalin, transferred to ethanol, dehydrated, and embedded in methyl methacrylate. Sections of 1-2 μm were stained with H & E and observed under a light microscope.

### Culture of Human Astrocytes and 3-Nitropropionic Acid Treatment

The SVG cell line used in this study was derived from human fetal astrocytes transformed with SV40 large T antigen (Major et al., J Biol Chem 278:13512-13519, 1985; Tornatore et al., Cell Transplant 5:145-163, 1996). Cultures of the human fetal astrocyte cell line (SVG) were maintained in Dulbecco's modified Eagle's medium with 2 mM L-glutamine, 10% fetal bovine serum, and streptomycin penicillin-fungizone solutions. Cells grown in 60-mm dishes were treated with 10 μM 3-nitropropionic acid (3NP) for 6, 12, and 24 hours at 37° C. and then fixed with paraformaldehyde (4%) for 15 minutes and blocked with 3% BSA at 4° C. overnight. The fixed cells were processed following standard techniques for immunofluorescence using VEGF and crystallin antibodies as indicated earlier.

### Morphometric Analysis of Retinal Thickness

Retinal thickness at E16/17 and for P3 and 12 were measured using AxioVision software (Zeiss). The results of regionally matched measurements from seven different retinas (n_{—}7) were analyzed and charted using Microsoft Excel.

### SDS-PAGE and Western-Blot Analysis

The eyes of freshly killed animals were enucleated and the retinae were dissected, rinsed in PBS and homogenized in 20 mM Tris, pH 7.1 containing Roche protease cocktail. Samples were spun in a microfuge at 14,000 r.p.m. for 15 minutes to remove nuclei and cell debris. The supernatant was removed, DNAse was added to it and incubated at room temperature for 30 minutes. The samples were then mixed with SDS sample preparation buffer and heated in a boiling waterbath for 2 minutes. The samples were run on 4-12% Bis-Tris Nu-PAGE gels (Invitrogen, Carlsbad, Calif., USA) stained with Gelcode Blue stain reagent (Pierce, Rockford, Ill., USA). For Western blotting, proteins were transferred to nitrocellulose membranes (Bio-Rad Laboratories, Hercules, Calif., USA), blocked with 5% milk diluent, and incubated with the primary antibody overnight at 4° C. HRP-conjugated secondary antibodies and Western lighting chemiluminescence reagent (Perkin Elmer Life Sciences, Wellesley, Mass., USA) were used for visualization. p27kip1 Polyclonal antibody (Stressgen) was used at 1:10,000 dilution. Anti GFAP mouse monoclonal (BD Pharmingen, San Diego, Calif., USA) was used at 1:500 dilution.

### Electroretinography (ERG) Studies on Nuc1 Homozygous and Age-Matched Wildtype Retinae

ERG was performed on 10 week old wild-type and Nuc1 mutant (both heterozygous and homozygous) rats according to a procedure adapted with minor modification from a previously published rodent protocol using gold wire loop electrodes (Lei, Doc Opthalmol 106(3):243-249, 2003). Maximum intensity was 0.65 log cd s/m2 and was attenuated over a 6-log range (step 1 log unit) with neutral-density filters (Eastman Kodak, Rochester, N.Y., USA). Amplitudes of dark-adapted ERG and b-waves and light-adapted ERG b-waves were measured and averaged between the two eyes of the same animal as one value in each animal for the purposes of statistical analysis. One-way analysis of variance and Student's t-test with Bonferroni correction were performed for comparing the Nuc1 mutant rats (both heterozygotes and homozygotes) and control groups (wild-type) at a given intensity, with P_{—}0.05 being assigned prospectively as the P value that would constitute statistical significance.

### Two Dimensional Electrophoresis and Mass Spectrometry

Two dimensional electrophoresis was performed essentially as previously described (Colvis and Garland, Arch Biochem Biophys 397:319-323, 2002). First dimension isoelectric focusing was performed on the Protean IEF System (BioRad) using 7 cm immobilized pH gradient strips (3-10 NL, Amersham Biosciences, Piscataway, N.J., USA). The strips were rehydrated in a solution of 7 M urea, 2 M thiourea, 4% CHAPS and 2.5 mg/ml DTT. Between 540 μg of protein was loaded and samples were focused for 16,000 volt hours.

The second dimension electrophoresis was performed using Inyitrogen's Minicell apparatus with 16% Tris glycine gels. Prior to the second dimension SDS-PAGE, the IPG strips were equilibrated for 15 minutes in 50 mM Tris, 6 M urea, 30% glycerol, 2% SDS (SDS equilibration solution) containing 10 mg/ml DTT and a second equilibration for 15 min in 40 mg/ml iodoacetamide-containing SDS equilibration solution. Protein spots were visualized with GelCode Blue Stain Reagent (Pierce).

Protein spots of interest were excised, minced and destained with 50% acetonitrile. Following extensive washing with 10 mM ammonium bicarbonate buffer, the proteins were trypsinized in gel overnight. The gel pieces were then dehydrated with 95% acetonitrile containing 1% formic acid. The extracted peptides were lyophilized and stored at _{—}20° C. until analyzed. The peptides were solubilized in 50% acetonitrile with 1% formic acid and allowed to dry on a steel support. When dry, a saturated solution of α-cyano-4-hydroxy-cinnamic acid was overlaid as the matrix. Masses of the peptides were determined by matrix-assisted laser desorption ionization time of flight mass spectrometry (MALDI-TOF) using a Voyager DE-STR (Applied Biosystems, Foster City, Calif., USA). Protein identification was by peptide mass fingerprinting using Protein Prospector search algorithm software and the SwissProt protein database.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A method for modulating a blood vessel in a subject in need thereof, the method comprising contacting a cell of the subject with a crystallin polypeptide, biologically active fragment, or mimetic thereof, thereby modulating the blood vessel.

2. The method of claim 1, wherein the method increases or decreases blood vessel formation relative to an untreated control tissue or organ.

3. The method of claim 1, wherein the method stabilizes or remodels a blood vessel in a tissue or organ relative to an untreated control tissue or organ.

4. A method for decreasing angiogenesis in a subject in need thereof, the method comprising contacting a cell of the subject with an agent that inhibits the expression or biological activity of a crystallin polypeptide.

5. A method of treating pathological neovascularization in a subject, the method comprising administering to the subject an agent that decreases angiogenesis in the subject, thereby treating pathological neovascularization in the subject.

6-8. (canceled)

9. A method for treating pathological ocular neovascularization in a subject in need thereof, the method comprising administering to the subject an effective amount of an agent that decreases angiogenesis in an ocular tissue thereby treating pathological ocular neovascularization in the subject.

10-21. (canceled)

22. A method for increasing blood vessel formation in a tissue or organ, the method comprising contacting a cell of the tissue or organ with a crystallin polypeptide thereby increasing blood vessel formation in the tissue or organ.

23. A method for stabilizing a blood vessel in a tissue or organ, the method comprising contacting a cell of the tissue or organ with a crystallin polypeptide, biologically active fragment, or mimetic thereof, thereby stabilizing a blood vessel in the subject.

24. A method for increasing blood vessel formation or stabilizing or remodeling a blood vessel in a tissue or organ, the method comprising contacting a cell of the tissue or organ with a nucleic acid molecule encoding a crystallin polypeptide, biologically active fragment, or mimetic thereof, thereby increasing blood vessel formation or stabilizing a blood vessel in a tissue or organ.

25-33. (canceled)

34. An inhibitory nucleic acid molecule that specifically binds at least a fragment of a nucleic acid molecule encoding a crystallin polypeptide and decreases the expression of the crystallin polypeptide.

35. (canceled)

36. An aptamer that specifically binds at least a fragment of a crystallin polypeptide and decreases a biological activity of the crystallin polypeptide.

37-38. (canceled)

39. A host cell comprising the nucleic acid molecule of claim 31.

40-41. (canceled)

42. A pharmaceutical composition for modulating a blood vessel in a subject comprising an effective amount of a crystallin polypeptide, biologically active fragment, or mimetic thereof in a pharmaceutically acceptable excipient.

43. A pharmaceutical composition for modulating a blood vessel in a subject comprising an effective amount of an inhibitory nucleic acid molecule of claim 34 that reduces the expression of a crystallin polypeptide in a pharmaceutically acceptable excipient.

44-48. (canceled)

49. A method of identifying a compound that modulates blood vessel formation, the method comprising contacting a cell that expresses a crystallin nucleic acid molecule with a candidate compound, and comparing the level of expression of the nucleic acid molecule in the cell contacted by the candidate compound with the level of expression in a control cell not contacted by the candidate compound, wherein an alteration in expression of the crystallin nucleic acid molecule identifies the candidate compound as a compound that modulates blood vessel formation.

50-51. (canceled)

52. A method of identifying a compound that modulates blood vessel formation, the method comprising contacting a cell that expresses a crystallin polypeptide with a candidate compound, and comparing the level of expression of the polypeptide in the cell contacted by the candidate compound with the level of polypeptide expression in a control cell not contacted by the candidate compound, wherein an alteration in the expression of the crystallin polypeptide identifies the candidate compound as a compound that modulates blood vessel formation.

53. A method of identifying a compound that modulates blood vessel formation, the method comprising contacting a cell that expresses a crystallin polypeptide with a candidate compound, and comparing the biological activity of the polypeptide in the cell contacted by the candidate compound with the level of biological activity in a control cell not contacted by the candidate compound, wherein an alteration in the biological activity of the crystallin polypeptide identifies the candidate compound as a candidate compound that modulates blood vessel formation.

54-61. (canceled)
